(19) United States
(12) Patent Application Publication
(10) Pub. No.: US 2026/0117421 A1
(43) Pub. Date: Apr. 30, 2026

(54) METHOD FOR PROFILING OF CELLS FROM GROUPS OF CELLS

(71) Applicants: Max-Planck-Gesellschaft zur Foerderung der Wissenschaften e.V., München (DE); Charité - Universitätsmedizin Berlin, Berlin (DE); The Regents of the University of California, Oakland, CA (US)

(72) Inventors: Stefanie GROSSWENDT, Berlin (DE); Alexander MEISSNER, Berlin (DE); Tobias CHRISTALLER, Berlin (DE); Philipp STACHEL-BRAUM, Berlin (DE); Zev GARTNER, San Francisco, CA (US); Brittany MOSER, San Francisco, CA (US); Eric CHOW, El Cerrito, CA (US)

(21) Appl. No.: 18/700,927

(22) PCT Filed: Oct. 14, 2022
(86) PCT No.: PCT/US2022/078120

§ 371 Date: Apr. 12, 2024

Related U.S. Application Data
(60) Provisional application No. 63/255,717, filed on Oct. 14, 2021.

(51) Int. Cl. C40B 20/04; C12N 15/10; C12Q 1/6806

(52) U.S. Cl. CPC C40B 20/04; C12N 15/1065; C12Q 1/6806

(57) ABSTRACT

The present invention provides a method for obtaining sequencing-based information from cells/nuclei, the method comprising (a) a first barcoding of cells and/or nuclei of a group of cells and/or nuclei comprising interacting cells with one or more group-specific barcode sequence(s) or a group-specific combination of one or more barcode sequence(s): (b) a second barcoding of nucleic acid molecules contained in and/or attached to individual cells and/or nuclei from said group of cells/nuclei with a cell/nucleus-specific barcode sequence; and (c) sequencing of the barcoded nucleic acid sequences.

### STATEMENT AS TO RIGHTS TO INVENTIONS MADE UNDER FEDERALLY SPONSORED RESEARCH AND DEVELOPMENT

This invention was made with government support under grant no. R01 GM135462 awarded by The National Institutes of Health. The government has certain rights in the invention.

The present invention provides a method for obtaining sequencing-based information from cells/nuclei, the method comprising (a) a first barcoding of cells and/or nuclei of a group of cells and/or nuclei comprising interacting cells with one or more group-specific barcode sequence(s) or a group-specific combination of one or more barcode sequence(s); (b) a second barcoding of nucleic acid molecules contained in and/or attached to individual cells and/or nuclei from said group of cells/nuclei with a cell/nucleus-specific barcode sequence; and (c) sequencing of the barcoded nucleic acid sequences.

In order to better understand the development, physiology and disease states of multicellular systems, it is pivotal to expand the scope of current cell atlas projects (e.g., the Human Cell Atlas (Rozenblatt-Rosen et al. (2017) Nature 550, 451-3)) from purely capturing single-cell RNA-sequencing (scRNA-seq) data to pairing this information with information on which cells in a tissue or blood sample for example are physically interacting with each other. However, despite the surge of scRNA-seq pipelines and protocols as exemplified by approaches such as Smart-seq2 (Picelli et al. (2014) Nat. Protoc. 9, 171-81), Drop-Seq (Macosko et al. (2015) Cell 161, 1202-14), 10× Genomics Chromium (Zheng et al. (2017) Nat. Commun. 8, 14049), or SPLIT-seq (Rosenberg et al. (2018) Science 360, 176-182), which are all able to simultaneously capture the gene activity of thousands of individual cells (and thus their cellular identity) at an ever-increasing throughput, all of these technologies have failed so far to provide additional information on which cells have been interacting with each other in the tissue and the information they send to and receive from their interacting cellular environment. This drawback can directly be attributed to the fact that in order to apply these types of sequencing technologies, samples containing the cells of interest have to be completely dissociated into single cell suspensions prior to sequencing library construction, a process during which physical cell-cell interactions within the tissue or any other sample are broken. This lack of spatial information prevents the identification of spatial expression patterns as well as external signals that influence a cell's behaviour and fate and therefore represents a major hurdle which has only been insufficiently addressed by a limited number of strategies in the prior art.

For example, cell-cell interactions have been predicted from scRNA-seq data based on the expression of compatible signalling molecules (Efremova et al. (2020) Nat. Protoc. 15, 1484-1506). However, such predictions suffer from several drawbacks in that they (a) only report which cells have the theoretical potential to interact with each other irrespective of their spatial location to one another, (b) rely on current, simplistic scientific knowledge which can render these predictions incomplete and incorrect, (c) in that they are based on assumptions and prior knowledge which impedes the de novo discovery of new cellular signalling patterns and cell neighbourhood gene expression programs, and (d) predictions are mostly based on the detection of expressed receptors and ligands in the averaged data of cell clusters rather than individual cells.

Alternatives that attempt to measure gene expression within the whole-tissue context focused on either of two further approaches. Spatial transcriptomics by sequencing RNA from tissue slices allows profiling of thousands of genes simultaneously, however since cell borders cannot be identified and/or defined, and therefore the produced data has no single-cell resolution and gene signals cannot be attributed to single cells (Stickels et al. (2021) Nat. Biotechnol. 39, 313-319). Spatial in situ hybridization on the other hand can in principle detect cell borders but only allows the profiling of a limited number of genes (Lohoff et al. (2021) Nat. Biotechnol, https://doi.org/10.1038/s41587-021-01006-2) and faces challenges in regards to cell segmentation. Both of these methods thus suffer from (a) reduced data depth which often requires integrating deeper standard scRNA-seq data with these spatial measurements, an approximation in which each cell of the scRNA data is mapped to where it resembles the spatial measurement, (b) the requirement for specialized instrumentation, (c) the fact that these techniques require data deconvolution or image segmentation steps, which are technically challenging and naturally imprecise, and (d) that there is no straightforward way for data integration with standard scRNA-seq data and/or data analysis.

Another approach relates to sequencing of cell doublets or triplets, which are isolated upon e.g. incomplete tissue dissociation. In one study, small cell groups had been further dissected manually with needles into single cells at the expense of very low throughput and impaired data quality (Boisset et al. (2018) Nat. Methods 15, 547-53), while otherwise the small cell ‘clumps’ are sequenced as a whole (Giladi et al (2020) Nat. Biotechnol. 38, 629-37; Manco et al. (2021) Nat. Commun. 12, 3074; Bahar Halpern et al. (2018) Nat. Biotechnol. 36, 962-70; Andrews et al. (2021) Nat. Methods 18, 912-20). However, the generated sequencing data is again not of single-cell resolution and must be compared against sequencing data generated from various a priori known reference cell types for the mixed cell type data to be properly computationally deconvoluted. Therefore, for each sample, relevant cell types first need to be identified this way and for each sequencing read in the mixed sequencing data, it needs to be estimated whether it is more likely to have originated from one or from the other cell (type). Thus, this computational deconvolution again represents an approximation which leaves a high degree of uncertainty and substantially reduces the resolution at which the gene activity of cells can be analyzed. In particular, gene expression programs that are caused by intercellular interactions cannot be measured precisely for each interacting cell using methods of the prior art.

In order to (a) increase the cellular throughput of next-generation sequencing campaigns or (b) capture additional information such as cell surface marker expression, single cells on their surface can physically be connected to an oligonucleotide encoded barcode which can subsequently be read out by sequencing. Experimentally, this has traditionally been achieved by (a) the direct integration of loose oligonucleotide complexes into plasma membranes of cells via hydrophobic anchor groups such as lipids or cholesterol (MULTI-seq; McGinnis et al. (2019) Nat. Methods 16, 619-26; WO 2020/010366), by (b) the use of a specific monoclonal antibody which is coupled to an oligonucleotide that binds to known cell surface proteins (cell hashing; Stoeckius et al. (2018) Genome Biol. 19, 224; WO 2018/144813), by (c) lectin-carbohydrate interactions, or by (d) multistep covalent procedures using e.g. click-tags; see e.g. Gehring et al. (2019) Nat Biotechnol 38, 35-38 (2020).

In view of the above, there is a need for the provision of improved means and methods for obtaining information about single cells as part of a group of cells, preferably cells in a tissue or in a multicellular model such as organoids.

The technical problem is solved by the embodiments provided herein and in particular as provided in the claims.

The present invention thus relates to, inter alia, the following items:
1. A method for obtaining sequencing-based information from cells/nuclei, the method comprising
   (a) a first barcoding of cells and/or nuclei of a group of cells and/or nuclei comprising interacting cells with one or more group-specific barcode sequence(s) or a group-specific combination of two or more barcode sequence(s);
   (b) a second barcoding of nucleic acid molecules contained in and/or attached to individual cells and/or nuclei from said group of cells/nuclei with a cell/nucleus-specific barcode sequence; and
   (c) sequencing of the barcoded nucleic acid sequences.
2. The method of item 1, wherein prior to step (a) the method comprises isolation of a group of cells/nuclei to be barcoded from a larger group of cells.
3. The method of item 2, wherein the larger group of cells is partially dissociated to obtain the group of cells and/or nuclei used in step (a).
4. The method of item 3, wherein partial dissociation is achieved by mechanical, chemical and/or enzymatic dissociation.
5. The method of item 4, wherein the obtained group of cells/nuclei comprises 2 to 100, 2 to 200, 2 to 300, 2 to 400, 2 to 500, 2 to 600, 2 to 700, 2 to 800, 2 to 900, 2 to 1000, 2 to 5000, 2 to 10000 or 2 to 20000 cells/nuclei.
6. The method of item 5, wherein the obtained group of cells/nuclei comprises 2 to 100 cells/nuclei.
7. The method of any one of items 1 to 6, wherein in step (a) one or more membrane-bound group-specific barcode sequence(s) is/are used.
8. The method of claim 7, wherein in step (a) one or more nucleic acid sequence(s) comprising a group-specific barcode sequence or a group-specific combination of two or more barcode-sequences are comprised or complexed in/with (a) modified primary oligonucleotide(s), preferably (an) oligonucleotide(s) with a hydrophobic modification and most preferably (a) lipid modified oligonucleotide(s) (LMO(s)), (a) cholesterol modified oligonucleotide(s) (CMO(s)) and/or (an) oligo(s) modified with tocopherol, phosphoramidite and/or another hydrophobic group.
9. The method of any one of items 1 to 8, wherein in step (a) one or more barcoding antibody(ies) are used or a group-specific combination of two or more barcoding antibody(ies), wherein the barcoding antibody or the group-specific combination thereof comprises a group-specific barcode, preferably wherein the barcoding antibody(ies) specifically bind to surface proteins of cells/nuclei comprised in the group of cells/nuclei.
10. The method of any one of items 1 to 9, wherein in step (a) one or more nucleic acid sequence(s) comprising a group-specific barcode sequence or a group-specific combination of two or more barcode-sequences are used, wherein the nucleic acid sequence(s) are internalized into cells/nuclei.
11. The method of any one of items 1 to 10, wherein in step (a) one or more nucleic acid sequence(s) comprising a group-specific barcode sequence or a group-specific combination of two or more barcode-sequences is/are used, wherein the nucleic acid sequence(s) are modified to bind covalently or non-covalently to cellular proteins, transmembrane proteins or extracellular components, such as the glycocalyx or extracellular matrix of cells/nuclei.
12. The method of any one of items 1 to 11, wherein in step (a) a combination of at least two of items 8 to 11 is used.
13. The method of any one of items 1 to 12, wherein the group-specific barcode sequence is contained in an oligonucleotide complex comprising at least one oligonucleotide on the cellular surface of individual cells/nuclei of the group of cells/nuclei.
14. The method of item 13, wherein the complex of item 13 further comprises a moiety for interlinking complexes of item 13.
15. The method of item 14, wherein the moiety for interlinking complexes can act directly, such as via photocrosslinking and/or chemical crosslinking groups, nucleophilic and electrophilic groups, multivalent molecules, protein-protein interactions, hybridizing bridge oligonucleotides, or indirectly via a multistep labeling procedure.
16. The method of item 14 or 15, further comprising a step of adding an agent inducing the covalent or non-covalent interlinking of complexes.
17. The method of any one of items 1 to 16, which is performed in the presence of an agent that increases barcoding efficiency, stability, and/or retention within and/or attached to the cells/nuclei, preferably leading to an optimized ion concentration in the cell suspension.
18. The method of any one of items 1 to 17, wherein the group of cells/nuclei is generated by
   (a) allowing individual cells to interact with each other; or
   (b) mechanical dissociation, laser dissection, enzymatic dissociation, chemical dissociation, and/or cutting preferably using a tissue cutting device for mechanical dissociation.
19. The methods of any one of items 1 to 18, wherein the group of cells/nuclei are isolated by manual isolation and/or automated isolation, using, for example, manual pipetting, laser capture microdissection, fluorescent-activated cell sorting (FACS), magnetic-activated cell sorting (MACS), buoyancy-activated cell sorting (BACS), flow cytometry, in particular large-object flow cytometry, density centrifugation, or dilution.
20. The method of item 19, wherein a fluorescent cellular reporter protein, fluorescent staining, fluorescent DNA staining and/or fluorescent antibody staining is used.
21. The method of any one of items 1 to 20, wherein the cells/nuclei prior to step (b), preferably prior to step (a), were subject to a non-natural influence, for example temperature, pressure, or ion concentration such as pH.
22. The method of any one of items 1 to 21, wherein prior to step (b), preferably prior to step (a), cells/nuclei were contacted with a chemical compound, a biological cell, a pathogen and/or a virus.
23. The method of any one of items 1 to 22, wherein the cells/nuclei have been obtained from an organism, embryo, fetus, tissue, organ, body fluid, tumor, and/or an in vitro culture.
24. The method of any one of items 1 to 23, wherein the cells comprise primary cells, genetically engineered cells, blood cells, somatic cells, natural or altered immune cells (such as CAR-T cells, CAR-NK cells, SynNotch CAR-T cells, T cells, engineered T cells, B cells, NK cells, macrophages, neutrophils, dendritic cells), virus-infected cells, pluripotent stem cells (iPS), embryonic stem cells, epiblast stem cells, adult stem cells, embryoid bodies, gastruloids, organoids, in vitro embryo models, and/or cell lines such as immortalized cells or cancer cell lines, and wherein the nuclei comprise nuclei obtained from one or more of said cells.
25. The method of any one of items 1 to 24, wherein sequencing is performed using Drop-seq, DroNc-seq, Nx1-seq, Seq-Well, sci-RNA-seq, scifi-RNAseq, sci-RNA-seq3, SCRB-seq, CEL-Seq, CEL-Seq2, SPLIT-seq, MARS-seq, Smart-seq/C1, C1-CAGE, RamDa-seq, Microwell-seq, Smart-seq2, Smart-seq3, bead-seq, LIBRA-seq, G&T-seq, Quartz-Seq, Perturb-seq, MULTI-seq, ChIP-seq, ATAC-seq, SNES, scBS-seq, scRRBS, Smart-RRBS, Drop-ChIP, scDam&T-seq, ScNaUmi-seq, DR-seq, CITE-seq, TotalSeq, CytoSeq, STRT-seq, STRT/C1, SUPeR-seq, REAP-seq, MATQ-seq, scM&T-seq, scMT-seq, scCAT-seq, Paired-seq, SNARE-seq, T-ATAC-seq, Slide-seq, RAGE-Seq, FB5P-seq, TEA-seq, SUGAR-seq, CRISP-seq, single-cell RNA-seq, single-cell sequencing using Fluent, multiplexed scRNA-seq via e.g. cell hashing or CellPlex, and/or CROP-seq.
26. An oligonucleotide complex comprising one or more oligonucleotide(s), a hydrophobic modification, the oligonucleotide or the oligonucleotide complex comprising an oligonucleotide linked to a hydrophobic entity, preferably lipid or cholesterol, a barcode sequence, and a moiety for interlinking complexes.
27. The oligonucleotide complex of item 26, wherein the moiety for interlinking complexes can act directly, such as via photocrosslinking and/or chemical crosslinking groups, nucleophilic and electrophilic groups, multivalent molecules, protein-protein interactions, hybridizing bridge oligonucleotides, or indirectly via a multistep labeling procedure.
28. The method of any of items 1 to 25, wherein the oligonucleotide or oligonucleotide complex of item 26 or 27 is used.
29. A kit comprising the oligonucleotide and/or the oligonucleotide complex of item 26 or 27, preferably together with instructions regarding the use of the method of any one of items 1 to 25.
30. The kit of item 29 further comprising an agent inducing the covalent or non-covalent interlinking of complexes.

In its broadest aspect, the present invention thus relates to a method for obtaining sequencing-based information from cells/nuclei, the method comprising (a) a first barcoding of cells and/or nuclei of a group of cells and/or nuclei comprising interacting cells with one or more group-specific barcode sequence(s) or a group-specific combination of two or more barcode sequence(s); (b) a second barcoding of nucleic acid molecules contained in and/or attached to individual cells and/or nuclei from said group of cells/nuclei with a cell/nuclei-specific barcode sequence; and (c) sequencing of the barcoded nucleic acid sequences.

The present inventors have surprisingly found that the combination of a group-specific barcoding and a subsequent cell-specific barcoding of cells and/or nuclei can provide information about their interactions and, in particular, the impact of such interactions on a genetic, transcriptomic and/or proteomic level. In this regard, the inventors have, for the first time, generated RNA-seq data that can be used to extract transcriptome signatures of physically interacting cells. Importantly, the barcoding strategy of the invention is conceptually different from (a) MULTI-seq which relies on more simple lipid- or cholesterol-modified oligonucleotide complex architectures (LMOs, CMOs) (McGinnis et al. (2019) Nat. Methods 16, 619-26) as well as (b) cell hashing which is based on cell surface antigen specific antibodies that are linked to a DNA barcode (Stoeckius et al. (2018) Genome Biol. 19, 224), in order to capture target antigen cell surface expression as well as the transcriptome of a cell by next-generation sequencing, with both, MULTI-seq and cell hashing being used for the unrelated purpose of sample multiplexing to label single-cells for joint processing of numerous samples at the same time. The barcoding strategy of the current invention also allows for substantially more stable and protease-resistant cell-membrane barcoding making it, for the first time, compatible with dissociations conditions and thus opening possibilities for group-specific barcoding with subsequent dissociation for single-cell sequencing applications.

The methods of the invention thus extend state-of-the-art scRNA-seq by group-specific, for example LMO/CMO-complex mediated barcoding, of cells and/or nuclei within or obtained from a cell group, either while cells are still attached to each other or after dissociation, thus enabling the unbiased cell/nuclei group-specific high-throughput genome, epigenome, transcriptome, proteome and/or metabolome profiling at single-cell resolution for the first time. In this regard, the methods of the present invention are sequencing-based, meaning that barcoded DNA/RNA comprised in or attached to a cell/nuclei is sequenced. With respect to proteome and/or metabolome analysis, indirect techniques may be employed, e.g. using nucleic acid tagged antibodies specific for certain proteins of a cell.

The herein provided methods can, inter alia, be used in combination with various sequencing approaches and are not particular limited to be used with one or more specific techniques. For example, but without being limited to this example, Drop-Seq or the Chromium platform commercialized by 10× Genomics, which is at the time of filing the most popular scRNA-seq platform, can be used. However, the methods of the invention may be adopted for the profiling of physically interacting cells in any other microfluidic or microplate-based platform in combination with methods or technologies that enable the sequencing of single cells/nuclei such as Drop-seq, DroNc-seq, Nx1-seq, Seq-Well, sci-RNA-seq, scifi-RNAseq, sci-RNA-seq3, SCRB-seq, CEL-Seq, CEL-Seq2, SPLIT-seq, MARS-seq, Smart-seq/C1, C1-CAGE, RamDa-seq, Microwell-seq, Smart-seq2, Smart-seq3, bead-seq, LIBRA-seq, G&T-seq, Quartz-Seq, Perturb-seq, MULTI-seq, ChIP-seq, ATAC-seq, SNES, scBS-seq, scRRBS, Smart-RRBS, Drop-ChIP, scDam&T-seq, ScNaUmi-seq, DR-seq, CITE-seq, TotalSeq, CytoSeq, STRT-seq, STRT/C1, SUPeR-seq, REAP-seq, MATQ-seq, scM&T-seq, scMT-seq, scCAT-seq, Paired-seq, SNARE-seq, T-ATAC-seq, Slide-seq, RAGE-Seq, FB5P-seq, TEA-seq, SUGAR-seq, CRISP-seq, single-cell RNA-seq, single-cell sequencing using Fluent, multiplexed scRNA-seq via e.g. cell hashing or CellPlex, and/or CROP-seq.

As such, the methods of the present invention can be used, inter alia, in tissue-scale, organ-scale and/or organism-scale single-cell/nuclei sequencing projects and/or developmental studies at the organ, tissue and/or organism level. The methods of the present invention may in that regard be used, inter alia, for the identification of autocrine, paracrine, juxtacrine and/or endocrine cellular communication signals that control cell identity such as desirable or less desirable cell signalling cascades, which can be therapeutically exploited by externally providing an organic or an inorganic compound/reagent that can act as an agonist or an antagonist such as immune checkpoint inhibitors for example. The methods of the invention may also be used to identify signaling events for diagnostic purposes and to identify binding preferences between cells, to interrogate the composition of cellular microenvironments, and to reveal aspects of overall tissue architecture. In this regard, the readout of the methods of the invention may be quantitative, e.g. the number of the cells/nuclei that have a certain characteristic (e.g. specific gene expression profile) amongst the profiled interacting cells/nuclei. The thus obtained information may be relevant e.g. in the case of engineered immune cells.

In further non-limiting embodiments of the present invention, the methods provided herein may be used to profile (a) the signalling inputs that a specific cell, or e.g. cell type, or nuclei of such a cell receives from its environment, in some instances the tumor microenvironment, (b) cells such as natural immune cells such as macrophages, dendritic cells, NK cells, T cells, B cells, tumor infiltrating lymphocytes, neutrophils or engineered immune cells such as CAR-T cells, SynNotch CAR-T cells, CAR-NK or engineered T-cells; (c) preferential cell-type specific virus infection and/or interactions between virus-infected cells and other host cells and/or (d) signalling cascades of virus-infected cells and/or cancer cells.

In another embodiment of the present invention, the methods of the invention may be used for the profiling of interacting natural, engineered, and/or synthetic cell and/or nuclei groups comprising prokaryotic cells and/or eukaryotic cells, for example for profiling cells comprised in the mammalian microbiome.

In this regard, the methods of the invention may comprise prior to step (a) a step of generating and isolating a group of cells and/or nuclei to be barcoded from a larger group of cells. Within the present invention, any technique known to the person skilled in the art may be employed to generate cell groups. For example, the group of cells/nuclei may be generated by allowing individual cells to interact with each other; or by mechanical dissociation, laser dissection, enzymatic dissociation, chemical dissociation, and/or cutting preferably using a tissue cutting device for mechanical dissociation. The group of cells/nuclei may be isolated by manual isolation and/or automated isolation, using, for example, manual pipetting, laser capture microdissection, fluorescent-activated cell sorting (FACS), magnetic-activated cell sorting (MACS), buoyancy-activated cell sorting (BACS), flow cytometry, in particular large-object flow cytometry, density centrifugation, or dilution.

In one embodiment of the invention, prior to step (a), a larger group of cells may be partially dissociated to obtain the group of cells and/or nuclei used in step (a) of the methods of the invention. Within the present invention, partial dissociation may be achieved by mechanical, chemical and/or enzymatic dissociation. It is preferred within the present invention that the obtained group of cells/nuclei comprises 2 to 100, 2 to 200, 2 to 300, 2 to 400, 2 to 500, 2 to 600, 2 to 700, 2 to 800, 2 to 900, 2 to 1000, 2 to 5000, 2 to 10000 or 2 to 20000 cells/nuclei, more preferably wherein the obtained group of cells/nuclei comprises 2 to 100 cells/nuclei. The cells may also be fixed, for example chemically fixed.

In certain embodiments of the invention, the methods of the invention comprise that in step (a) one or more group-specific barcode sequence(s) is/are used or a group-specific combination of two or more barcode sequence(s), wherein the barcode sequence(s) is/are (a) modified primary oligonucleotide(s), preferably (an) oligonucleotide(s) with a hydrophobic modification and most preferably (a) lipid modified oligonucleotide(s) (LMO), cholesterol modified oligonucleotide(s) (CMO) and/or (an) oligo(s) modified with tocopherol, phosphoramidite and/or another hydrophobic group.

In certain embodiments of the invention, the methods of the invention comprise that in in step (a) one or more nucleic acid sequence(s) comprising a group-specific barcode sequence or a group-specific combination of two or more barcode-sequences are comprised or complexed in/with (a) modified primary oligonucleotide(s), preferably (an) oligonucleotide(s) with a hydrophobic modification and most preferably (a) lipid modified oligonucleotide(s) (LMO(s)), (a) cholesterol modified oligonucleotide(s) (CMO(s)) and/or (an) oligo(s) modified with tocopherol, phosphoramidite and/or another hydrophobic group.

In a further embodiment, a first barcoding of cells and/or nuclei of a group of cells and/or nuclei comprising interacting cells with one or more group-specific barcode sequence(s) may be done using sequences for barcoding where the combination of said sequences is group-specific.

In the methods of the invention, it is not required that all cells of a cell group receive all barcodes or a barcode combination. It may be sufficient to identify some of the barcodes if the recovered barcode combination still allows identification of group of cells.

However, the methods of the present invention are not limited to the use of membrane-bound group-specific barcode sequences. Rather, various technologies for group-specific barcoding can be employed within the present invention. In this regard, the skilled person is aware of various techniques used for barcoding nucleic acid molecules. While such techniques may be valuable, the methods of the present invention differ from any of such approaches by focusing on cell group-specific barcoding, namely approaches resulting in an exclusive barcoding of cells being part of a specific group of cells. The present inventors have also found that for a cell of a group of cells not all barcodes of the group-specific barcode combination need to be recovered to assign the cell to the cell group it originated from. If the barcode combination is incompletely identified for an individual cell, this can still suffice for reliable assignment.

In some embodiments of the invention, in step (a) one or more barcoding antibody(ies) are used or a group-specific combination of two or more barcoding antibody(ies), wherein the barcoding antibody or the group-specific combination thereof comprises a group-specific barcode, preferably wherein the barcoding antibody(ies) specifically bind to surface proteins of cells/nuclei comprised in the group of cells/nuclei, preferably after complete dissociation, in particular after enzymatic dissociation, in particular when proteases are used for dissociation.

In further embodiments of the invention, in step (a) of the methods of the invention one or more nucleic acid sequence(s) each alone or in combination comprising a group-specific barcode sequence may be used, wherein the nucleic acid sequence(s) are internalized into cells/nuclei. In further embodiments of the invention, in step (a) of the methods of the invention one or more nucleic acid sequence(s) comprising a group-specific barcode sequence or a group-specific combination of two or more barcode-sequences is/are used, wherein the nucleic acid sequence(s) are internalized into cells/nuclei. This has been, inter alia, used in sci-Plex (Srivatsan et al. 2020), where poly-adenylated, single-stranded oligos were used to label nuclei; the cells were lysed and the oligos entered the permeabilized nuclei.

In further embodiments of the invention, in step (a) one or more nucleic acid sequence(s) comprising a group-specific barcode sequence or a group-specific combination of two or more barcode-sequences is/are used, wherein the nucleic acid sequence(s) are modified to bind covalently or non-covalently to cellular proteins, transmembrane proteins or extracellular components, such as the glycocalyx or extracellular matrix of cells/nuclei. Further, LMOs may contain lipids that bind to the membrane, or antibodies that bind to surface-proteins or other modifications/devices may be found to barcode other components of the cell surface, such as sugars of the glycocalyx. For example, a biotinylated lectin has been used to detect surface N-glycans using an anti-biotin antibody; see e.g. Kearney et al. (2021), Science Advances 7, Issue 8.

It is also envisaged within the present invention to use combinations of the various approaches for targeting cells or parts thereof. Accordingly, in step (a) a combination of at least two of the approaches disclosed above may be used.

Barcoding of cells/nuclei from cell groups may be accomplished with a variety of barcoding strategies. In addition to the barcoding based on hydrophobic modifications that integrate into the cell membrane, additional examples are provided and envisaged herein.

Within the present invention, the complexes containing one or more group-specific barcode sequence(s) may be inter-linked on the surface of individual cells/nuclei of the group of cells/nuclei, preferably directly via photo-crosslinking groups, nucleophilic and electrophilic groups, through multistep covalent procedures such as click-tags, multivalent molecules such as lectins, streptavidin, PEG or antibodies, hybridizing bridge oligonucleotides, or indirectly via a multistep labeling procedure. Accordingly, provided herein is an oligonucleotide complex comprising one or more oligonucleotide(s), a hydrophobic modification, the oligonucleotide or the oligonucleotide complex comprising an oligonucleotide linked to a hydrophobic entity, preferably lipid or cholesterol, a barcode sequence, and a moiety for interlinking complexes. In a particular embodiment, the moiety for interlinking complexes can act directly, such as via photocrosslinking and/or chemical crosslinking groups, nucleophilic and electrophilic groups, multivalent molecules, protein-protein interactions, hybridizing bridge oligonucleotides, or indirectly via a multistep labeling procedure.

In further embodiments of the invention, barcoding efficiency, stability, and/or retention within and/or attached to the cells/nuclei, may be enhanced using for example an agent such as a chelating agent such as phosphonate, NTA (Nitrilotriacetic acid), EDTA (Ethylenediamine-tetraacetic acid Disodium salt), EGTA (Ethyleneglycol-O, O′-bis(2-aminoethyl)-N,N,N′,N′-tetraacetic acid), HEDTA (N-(2-Hydroxyethyl)ethylenediamine-N,N′,N′-triacetic acid Trisodium salt).

In further embodiments of the invention, the presence of the agent increasing barcoding efficiency, stability, and/or retention within and/or attached to the cells/nuclei leads to an optimized ion concentration in the suspension.

Within the methods of the present invention, the group of cells/nuclei may be generated by allowing individual cells to interact with each other; or by mechanical dissociation, laser dissection, enzymatic dissociation, chemical dissociation, and/or cutting preferably using a tissue cutting device for mechanical dissociation.

Within the methods of the present invention, the group of cells/nuclei may be isolated by manual isolation and/or automated isolation, using, for example, manual pipetting, laser capture microdissection, fluorescent-activated cell sorting (FACS), magnetic activated cell sorting (MACS), buoyancy-activated cell sorting (BACS), flow cytometry, in particular large-object flow cytometry, density centrifugation, or dilution.

Within the methods of the present invention a fluorescent cellular reporter protein, fluorescent staining, fluorescent DNA staining and/or fluorescent antibody staining may be used, for example in order to label cells/nuclei comprised in the group of cells used in the methods of the invention.

In a further embodiment of the invention, the cells/nuclei prior to step (b), preferably prior to step (a) are subject to a non-natural influence, for example temperature, pressure, ion concentration such as pH. In this regard, the term “non-natural” relates to a state where a deviation of what would be considered “natural” by the person skilled in the art is observed.

In a further embodiment of the invention, cells/nuclei prior to step (b), preferably prior to step (a) are contacted with a chemical, inorganic and/or organic compound, a biological cell, a pathogen and/or a virus.

Within the present invention, it is disclosed that the cells/nuclei have been obtained from a tissue, organ, body fluid, tumor, and/or an in vitro culture.

The methods of the present invention may, inter alia, be combined with single-cell sequencing technologies known to the person skilled in the art, such as e.g. ATAC-seq for the assessment of chromatin accessibility and/or with single-cell genome sequencing and/or with single-cell bisulfite sequencing for the extraction of DNA-methylation/epigenetic sequence information and/or with single-cell ChIP-sequencing for the assessment of protein-DNA interactions or histone modifications. The methods of the present invention may also be combined with barcoded entities, such as antibodies or nucleic acid molecules prior to sequencing library construction for an integrated readout of cell surface proteins and/or any other relevant DNA or RNA oligonucleotide encoded sequence information of interacting cells. Such barcoded entities may or may not correspond to the barcoding antibody(ies) or nucleic acid sequence(s) as used in the methods of the present invention.

Within the present invention, the cells may comprise any kind of cells, such as primary cells, engineered cells, blood cells, somatic cells, natural or altered immune cells (such as CAR-T cells, CAR-NK cells, SynNotch CAR-T cells, T cells, engineered T cells, B cells, NK cells, macrophages, neutrophils, dendritic cells), virus-infected cells, pluripotent stem cells (iPS), embryonic stem cells, epiblast stem cells, adult stem cells, embryoid bodies, gastruloids, organoids, in vitro embryo models, and/or cell lines such as immortalized cells or cancer cell lines or entirely artificial cell lines.

Within the present invention, sequencing may be performed using any method known to the person skilled in the art. The following methods are thus merely provided as exemplary methods. Sequencing may be performed using, for example, Drop-seq, DroNc-seq, Nx1-seq, Seq-Well, sci-RNA-seq, scifi-RNAseq, sci-RNA-seq3, SCRB-seq, CEL-Seq, CEL-Seq2, SPLIT-seq, MARS-seq, Smart-seq/C1, C1-CAGE, RamDa-seq, Microwell-seq, Smart-seq2, Smart-seq3, bead-seq, LIBRA-seq, G&T-seq, Quartz-Seq, Perturb-seq, MULTI-seq, ChIP-seq, ATAC-seq, SNES, scBS-seq, SCRRBS, Smart-RRBS, Drop-ChIP, scDam&T-seq, ScNaUmi-seq, DR-seq, CITE-seq, TotalSeq, CytoSeq, STRT-seq, STRT/C1, SUPeR-seq, REAP-seq, MATQ-seq, scM&T-seq, scMT-seq, scCAT-seq, Paired-seq, SNARE-seq, T-ATAC-seq, Slide-seq, RAGE-Seq, FB5P-seq, TEA-seq, SUGAR-seq, CRISP-seq, single-cell RNA-seq, single-cell sequencing using Fluent, multiplexed scRNA-seq via e.g. cell hashing or CellPlex, inCITE-seq and/or CROP-seq.

Also provided by the methods of the present invention is the analysis of interacting cells in the blood or lymph, for example the analysis of interacting cells containing at least one virus-infected cell or a tumor cell, preferably a circulating and/or disseminated tumor cell.

A potential outcome of the methods of the present invention may be the gain of information about the effect of added components, such as for example chemical molecules, biological molecules, cells and/or environmental factors on cells comprised in a group of cells, for example for assessing the effectiveness of a chemical molecule on the treatment of a disease. In this regard, the disease may inter alia be an autoimmune disease such as Rheumatoid arthritis, Systemic lupus erythematosus, Inflammatory bowel disease, Multiple sclerosis, Type 1 diabetes mellitus, Guillain-Barre syndrome, Chronic inflammatory demyelinating polyneuropathy, Psoriasis, Graves' disease, Hashimoto's thyroiditis, Myasthenia gravis, or Vasculitis. Further comprised are viral infections caused by Adenovirus, Coxsackievirus, Cytomegalovirus, Epstein-Barr virus, Hepatitis A virus, Hepatitis B virus, Hepatitis C virus, Herpes simplex virus type 1, Herpes simplex virus type 2, HIV, Human coronavirus 229E, Human coronavirus NL63, Human coronavirus OC43, Human coronavirus HKU1, Human herpesvirus type 8, Human papillomavirus, Influenza virus, Measles virus, Middle East respiratory syndrome-related coronavirus, Mumps virus, Parainfluenza virus, Poliovirus, Rabies virus, Respiratory syncytial virus, Rubella virus, Severe acute respiratory syndrome coronavirus, Severe acute respiratory syndrome coronavirus 2, or Varicella-zoster virus. Further comprised are cancers such as Adenoid cystic carcinoma, Adrenal gland tumor, Amyloidosis, Anal Cancer, Appendix cancer, Astrocytoma, Ataxia-Telangiectasia, Beckwith-Wiedemann Syndrome, Bile duct cancer, Birt-Hogg-Dubé syndrome, Bladder cancer, Bone cancer, Brain stem glioma, Brain tumor, Breast cancer, Carney complex, Central Nervous System Tumors, Cervical cancer, Colorectal cancer, Cowden syndrome, Craniopharyngioma, Desmoid tumor, Desmoplastic infantile ganglioglioma, Ependymoma, Esophageal cancer, Ewing sarcoma, Eye cancer, Eyelid cancer, Familial adenomatous polyposis, Familial GIST, Familial malignant melanoma, Familial pancreatic cancer, Gallbladder cancer, Gastrointestinal stromal tumor, Germ cell tumor, Gestational trophoblastic disease, Head and neck cancer, Hereditary breast and ovarian cancer, Hereditary diffuse gastric cancer, Hereditary leiomyomatosis and renal cell cancer, Hereditary mixed polyposis syndrome, Hereditary pancreatitis, Hereditary papillary renal carcinoma, HIV/AIDS related cancer, Juvenile polyposis syndrome, Kidney cancer, Lacrimal gland tumor, Laryngeal and hypopharyngeal cancer, Acute Lymphoblastic Leukemia, Acute Lymphocytic Leukemia, Acute Myeloid Leukemia, B-cell Prolymphocytic Leukemia and Hair Cell Leukemia, Chronic Lymphocytic Leukemia, Chronic Myeloid Leukemia, Chronic T-Cell Lymphocytic Leukemia, Eosinophilic Leukemia, Li-Fraumeni syndrome, Liver cancer, Non-small cell lung cancer, Small cell lung cancer, Hodgkin lymphoma, Non-Hodgkin lymphoma, Lynch syndrome, Mastocytosis, Medulloblastoma, Melanoma, Miningioma, Mesothelioma, Multiple Endocrine Neoplasia Type 1, Multiple Endocrine Neoplasia Type 2, Multiple Myeloma, MUTYH-Associated Polyposis, Myelodysplastic syndromes, Nasal cavity and paranasal sinus cancer, Nasopharyngeal cancer, Neuroblastoma, Neuroendocrine tumor, Neurofibromatosis Type 1, Neurofibromatosis Type 2, Nevoid basal cell carcinoma syndrome, Oral and oropharyngeal cancer, Osteosarcoma, Ovarian cancer, Pancreatic cancer, Parathyroid cancer, Penile cancer, Peutz-Jeghers syndrome, Pheochromocytoma, Paraganglioma, Pituitary gland tumor, Pleuropulmonary blastoma, Prostate cancer, Retinoblastoma, Rhabdomyosarcoma, Salivary gland cancer, Kaposi sarcoma, Skin cancer, Small bowel cancer, Stomach cancer, Testicular cancer, Thymoma and Thymic carcinoma, Thyroid cancer, Tuberous sclerosis complex, Uterine cancer, Vaginal cancer, Von Hippel-Lindau syndrome, Vulvar cancer, Waldenstrom macroglobulinemia, Werner syndrome, Wilms tumor, Xeroderma pigmentosum.

As demonstrated in the appended examples, the methods of the present invention constitute the first use of cell group barcoding for single-cell transcriptome sequencing of interacting cells. The present invention also provides the first use of a stabilized LMO barcoding strategy for the membrane-barcoding of cell groups. While LMOs/CMOs have recently been described for multiplexing of samples for scRNA-seq, their use has not been suggested for single-cell sequencing of interacting cells within a cell group. The invention as described herein thus relates to, inter alia, barcoding of groups of physically attached cells as being compatible with standard single-cell sequencing library preparation protocols (e.g. 10× Genomics' 3′ kit or alternative protocols such as DropSeq). To the inventors' knowledge, there is no other single-cell sequencing method using a stabilized oligo, preferably LMO/CMO, barcoding strategy that would enable the single-cell sequencing of interacting cells prior to the present invention. For the relevant methods provided herein, it was demonstrated that the use of a stabilized complex is effective for preparing single-cell transcriptome libraries. One exemplary experimental workflow for the single-cell sequencing of interacting cells is illustrated in FIG. 6, and its application to the profiling of complex tissues is demonstrated in FIG. 8.

In summary, in the methods of the present invention, it is preferred that a novel stabilized LMO/CMO complex architecture is used to introduce an oligonucleotide-encoded barcode into the membrane of physically interacting cells within a cell group via a hydrophobic group prior to complete dissociation of interacting cells and single-cell sequencing library preparation in order to profile nucleic acid sequences of physically interacting cells. This demonstrated use makes plausible that the use of alternative barcoding strategies such as those characterized above is feasible in order to reach the effect characterized above. In an alternative embodiment of the invention, the barcode may be introduced after dissociation of cells of a cell group, wherein cells of a cell group are contained in the same compartment.

Thus, in general, the invention relates to a method for obtaining sequencing-based information from cells/nuclei. Sequencing-based information of cells/nuclei is generally obtained by sequencing of nucleic acid molecules being contained in or attached to cells, wherein said nucleic acid molecules may comprise RNA and/or DNA. The term “sequence” refers to an oligonucleotide or any portion of the nucleic acid molecule that is at least two or more units (nucleotides) long. The term can also be used as a reference to the nucleic acid molecule itself or a relevant portion thereof.

Nucleic acid sequence information generally relates to the succession of nucleotide bases in the respective oligonucleotide(s), in particular DNA and/or RNA, in particular DNA and/or RNA being contained in or attached to the cells/nuclei as in the methods of the present invention. For example, if the nucleic acid molecule contains canonical bases adenine, guanine, cytosine, thymine and/or uracil, naturally occurring modified analogs thereof or synthetic chemical analogs thereof, the nucleic acid molecule sequence can be represented by a corresponding string of letters A, G, C, T or U etc. Such nucleic acid molecules may be sequenced using the methods of the present invention.

As indicated above, nucleic acid molecule sequence information of the present invention may, inter alia, also cover synthetic and/or natural non-canonical nucleotides, preferably 5-methylcytosine.

In a first step, the methods of the invention may comprise a first barcoding of cells/nuclei of a group of cells comprising interacting cells with one or more group-specific barcode sequence(s), for example with a membrane-bound barcode sequence. The method may also comprise in step (a) the use of a group-specific combination of one or more barcode sequence(s).

The methods of the invention may further comprise isolating said group of cells to be barcoded from a larger group of cells into a first reaction compartment.

Within the present invention, the group of cells may be generated and/or isolated by allowing individual cells to interact with each other or mechanical dissociation, laser dissection, enzymatic dissociation, chemical dissociation, and/or cutting preferably using a tissue cutting device for mechanical dissociation.

The group of cells/nuclei may be isolated into a first reaction compartment by manual isolation and/or automated isolation, using, for example, manual pipetting, laser capture microdissection, fluorescent-activated cell sorting (FACS), magnetic-activated cell sorting (MACS), buoyancy-activated cell sorting (BACS), flow cytometry, in particular large-object flow cytometry, density centrifugation, or dilution.

Within the present invention a sample may comprise a known or unknown portion or quantity of an organism, organ, blood, lymph, normal tissue, diseased tissue or cell group comprising cells. Cells may be eukaryotic cells obtained from, inter alia, humans, non-human primates, horses, alpacas, llamas, cows, pigs, dogs, cats, rabbits, gerbils, armadillos, chinchillas, guinea pigs, mice, rats, hamsters, fish, amphibians, reptiles, birds, insects, plants or cell lines. Furthermore, cells may be healthy or diseased cells.

The cells may be present in various states and may be obtained from samples of various states or origins.

For example, in one embodiment, the cells may be obtained from in vitro cultures or fresh, chemically fixed, or frozen samples.

Samples may also be subject to fixation, for example formalin/formaldehyde-fixation or mild fixation using dithiobis(succinimidyl propionate) (DSP).

Within the present invention, the cells may be of any origin as long as the cells comprise nucleic acid molecules comprising RNA and/or DNA. For example, the cells may be cell lines, embryonic stem cells, embryonic bodies, pluripotent stem cells (iPS), somatic cells, blood cells (i.e., T cells, B cells, NK cells, macrophages, neutrophils, dendritic cells) or genetically engineered variants thereof transiently and/or stably expressing recombinant proteins, the source of which is exogenously supplied in the form of linear and/or circularized DNA and/or RNA oligonucleotides via genetic engineering, transduction, transfection, lipofection, transformation, infection, micro injection, a gene gun/biolistic particle delivery system and the like such as engineered T-cells, CAR-T-cells, CAR-NK cells, SynNotch CAR-T-cells or SUPRA CAR-T-cells. Cells may further undergo natural differentiation, artificially induced reprogramming or transdifferentiation.

Further envisaged to be used in the methods of the present invention are virus infected cells, cancer cells, tumor associated macrophages and/or tumor infiltrating lymphocytes. As such, the methods of the present invention may, inter alia, be used in immune oncology (CAR-T-cells, CAR-NK cells, SynNotch CAR-T-cells, SUPRA CAR-T-cells, engineered T-cells, TILs, bispecific engagers, BiTEs, immune checkpoint blockade, cancer vaccines delivered as mRNA), molecularly targeted cancer therapy, the dissection of drug resistance and/or toxicity mechanisms and/or in target discovery and/or validation.

In further embodiments, the cells may be obtained from biological material used in forensics, reproductive medicine, regenerative medicine and/or immune oncology.

Accordingly, the cells may be cells derived from a tumor, a tumor biopsy, blood, bone marrow aspirates, lymph nodes and aspirates thereof and/or cells obtained from a tissue or organ, lymph, a micro-dissected tissue, a blastomere or blastocyst of an embryo, embryonic or fetal tissue, a sperm cell, cells obtained from amniotic fluid, or cells obtained from buccal swabs and/or nasopharyngeal swabs. Tumor cells may preferably be disseminated tumor cells, circulating tumor cells or cells from tumor biopsies. Furthermore, blood cells may preferably be peripheral blood cells or cells obtained from umbilical cord blood. It is particularly preferred that the oligonucleotides comprised in the cells represent the genome, epigenome, transcriptome or proteome of the cells.

However, it is also envisaged to use non-eukaryotic cells within the present invention such as e.g. cells from prokaryotes, archaea, fungi, plants, ciliates, flagellates, trichomonads, microsporidia, diplomonads, slime molds, entamoebae, haloarchaea, methanosarcina, methanobacterium, methanococcus, thermococcus, thermoproteus, pyrodicticum, chloroflexi, gram positives, spirochetes, proteobacteria, cyanobacteria, planctomyces, bacteroides cytophaga, thermotoga, aquifex and/or the microbiota constituting the microbiome of higher eukaryotes.

In another embodiment of the present invention, cells prior and/or subsequent to the first barcoding step, in particular step (a) of the methods of the invention, may be subjected to a non-natural influence, for example to changes in buffer composition/pH and/or temperature.

In another embodiment, cells prior and/or subsequent to the first barcoding step, in particular step (a) of the methods of the invention, may be contacted with a chemical, inorganic and/or organic compound, a biological cell, a pathogen, and/or a virus.

Physical attachment of cells within the scope of the present invention may be a consequence of cell-cell interaction and may thus be established by interactions via the extracellular matrix, protein-matrix interactions, protein-protein interactions, preferably involving surface receptors, adhesion molecules, gap junction proteins, and the like. Further, nucleic acid molecules produced and exposed by cells on their cell surface can mediate or participate in cell interactions. Within a group of cells, it is not required that each cell is in direct physical contact with every other cell of the group of cells.

Partial dissociation in the sense of the present invention refers to the incomplete dissociation of tissue or similar samples comprising cells into smaller groups of cells comprising at least two or more cells, preferably 2 to 100, 2 to 200, 2 to 300, 2 to 400, 2 to 500, 2 to 600, 2 to 700, 2 to 800, 2 to 900, 2 to 1000, 2 to 5000, 2 to 10000 or 2 to 20000 cells/nuclei, more preferably wherein the obtained group of cells/nuclei comprises 2 to 100 cells/nuclei, by, inter alia, mechanical, enzymatic and/or chemical dissociation. The desired number of cells/nuclei in the group may depend on the question to be addressed. The skilled person is aware of suitable methods to provide groups of cells/nuclei of said numbers.

Mechanical dissociation may be carried out by application of physical force using a tool such as scissors, a razor blade, a glass mortar, a spatula, a pestle, a laser, and/or a grid in a process comprising the acts of cutting, crushing, dicing, scraping, dissecting, or grinding and/or combinations thereof. In some instances of the present invention, mechanical dissociation may preferably be achieved by an automated tissue homogenizer, tissue cutter, ultrasonication, laser-assisted cutting or by the repeated exposure of tissues and/or tissue pieces to shear-forces preferably by the act of repeated pipetting. Combinations of the aforementioned mechanical dissociation methods may in some instances be preferred. It should be noted that while mechanical dissociation is quick and especially beneficial when working with loosely associated tissue/cell samples, it may also lead to inconsistent results between users and important parameters such as cell yields and cell viabilities may thus vary widely.

In another embodiment of the present invention, enzymes may be used for partial dissociation. In such an instance, the use of collagenase I, collagenase II, collagenase III, collagenase IV, trypsin, TrypLET, dispase, Accutase, Liberase™ and/or hyaluronidase or a combination thereof may be preferred depending on the sample to be processed. Dissociation enzymes that are active at a range of temperatures may be used, including cold-active proteases that may be used for dissociations at a variety of temperatures, including lower temperatures. Notably, while enzymatic dissociation may be used for more compact tissues, it may also inadvertently modify proteins on the surface of target cells, thus altering their function or how a flow cytometer in combination with a specific barcoded antibody may identify them.

In another embodiment of the present invention, chemical dissociation may be used for partial dissociation. In such an instance, the single or combined use of cation chelating agents, preferably the use of calcium cation chelating agents such as EDTA, EGTA, HEDTA, dipicolin acid (DPA), nitrilotriacetic acid (NTA), β-alanine diacetate (ADA), citric acid and more preferably EDTA may be recommended which disrupt calcium-dependent cell adhesion molecules (CAMs), preferably integrins, cadherins and/or selectins. Typically, chemical dissociation may be used for more gentle forms of dissociation, and it may result in high cell viability.

In another embodiment of the present invention, it may be preferred to use combinations of mechanical, enzymatic and/or chemical dissociation methods for partial dissociation of samples.

In another embodiment of the present invention the group of cells may be obtained by, inter alia, in vivo or in vitro assembly of single cells that interact with each other or by direct isolation of cell doublets or cell groups (two or more cells).

In some instances of the present invention, it may be preferred to separate groups of cells of a defined size from single cell and/or tissue debris prior to or in the course of isolating the group of cells into the first reaction compartment. Separation may be achieved by means of a cell strainer for example that exhibits a mesh size that is suitable for enriching groups of cells of a desired size range, preferably a cell strainer with a pore size of 1, 3, 5, 8, 10, 15, 20, 30, 40, 50, 60, 70, 85, 100, 150, 200, 300, or 400 μm.

In another embodiment of the present invention, it may be preferred that the group of cells is isolated into the first reaction compartment, wherein the group of cells may be completely dissociated into single cells prior to barcoding using the membrane-bound barcode sequence.

In another embodiment, the first reaction compartment of the present invention may be a single well of a 6, 12, 24, 96, 384, 1536, or 3456-well plate, a well of a microwell array or microwell plate, a droplet or a plastic tube.

Multiple cell groups may be combined into one reaction compartment, if a split-pool group-specific and/or cell/nuclei-specific barcoding approach (cell-specific barcoding disclosed by Rosenberg et al. (2018) Science 360, 176-182) is followed, for example to increase the throughput of cell-group specific barcoding and/or of cell-specific barcoding. Accordingly, within the present invention cells of a cell group may be barcoded while cells are interacting within a group of cells or after cells of a group have been separated.

In order to perform the first barcoding of the group of cells, preferably interacting cells, with a group-specific barcode or a combination of barcode sequences, for example membrane-bound barcode sequences, modified oligonucleotides, preferably oligonucleotides with a hydrophobic modification and most preferably lipid modified oligonucleotides (LMOs) and/or cholesterol modified oligonucleotides (CMOs) may be used. As described above, various alternative group-specific barcoding strategies may be employed within the methods of the present invention.

Barcoding of the cell group with the group-specific barcode sequence or combinations of barcode sequences in the sense of the present invention describes, in one embodiment, the act of attaching copies of the same distinguishable (set of) oligonucleotide barcode sequence(s) to cells being comprised in the group of cells, for example by membrane insertion of the hydrophobic/lipid or cholesterol modification. Thus, cells being contained in two different groups of cells for example, carry two different barcodes or two different combinations of barcodes. However, within the present invention, the group-specific barcode may also be introduced by, for example one or more antibody(ies), wherein each barcoding antibody is attached to a barcode sequence, preferably wherein the antibody(ies) specifically bind to surface proteins of cells/nuclei comprised in the group of cells/nuclei. The group-specific barcode sequence or combination of barcode sequences may also be internalized into cells/nuclei. In a further embodiment, one or more nucleic acid sequence(s) comprising a group-specific barcode sequence or combination of barcode sequences may be used, wherein the nucleic acid sequence(s) are modified to bind to extracellular components or the glycocalyx of cells.

Within the present invention, combinations of the group-specific barcoding strategies described above may be used.

The term “barcode” in accordance with the invention is to be understood as a string of nucleotides with a defined length and position within an oligonucleotide that is known or may not be known, wherein each position has an independent and equal probability of being a nucleotide. The nucleotides of a barcode sequence can be any of the nucleotides, for example G, A, C, T, U or chemical analogs thereof, in any order, wherein: G is understood to represent guanylic nucleotides, A adenylic nucleotides, T thymidylic nucleotides, C cytidylic nucleotides and U uracylic nucleotides.

Oligonucleotide modifications in the sense of the present invention may comprise, inter alia, hydrophobic, lipid and/or cholesterol modifications.

Hydrophobic modifications may comprise, inter alia, organic compounds such as benzene derivatives (including fused or linked aromatic groups), or branched or unbranched alkanes or cyclic alkenes, alkynes or derivatives thereof comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 carbon atoms for example which dissolve in nonpolar solvents, and combinates of such modifications.

Lipid modifications may comprise, inter alia, unsaturated and saturated fatty acids, such as lignoceric acid or palmitic acid, glycerolipids, glycerophospholipids, sphingolipids, sterols, prenols, saccharolipids or polyketides.

Cholesterol modifications may also comprise derivatives and stereoisomers thereof that arise from its eight stereocenters.

In some instances, modified primary oligonucleotides may also be modified with an azide, alkyne, 6-FAM and/or 5-TAMRA functional group in order to enable click chemistry.

Modified oligonucleotide complexes of the present invention may comprise two or more (partially) complementary DNA oligonucleotides, at least one of them may comprise at least one modification from the aforementioned list of modifications (hydrophobic, lipid or cholesterol), a group-specific barcode sequence, a primer binding site, and a capture sequence, preferably a polyA sequence.

Furthermore, modified oligonucleotides of the present invention may comprise oligonucleotides with a length of at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides whereas said nucleotides may also comprise nucleotide modifications such as phosphorothioated bases, phosphorothioated 2′ O-methyl bases, locked nucleic acids, 2-MethoxyEthoxy A, 2-MethoxyEthoxy MeC, 2-MethoxyEthoxy G, 2-MethoxyEthoxy T, 2′ Fluoro C, 2′ Fluoro A, 2′ Fluoro G, 2-Aminopurine, 5-Bromo dU, deoxyUridine, 2,6-Diaminopurine, Dideoxy-C, deoxylnosine, Hydroxymethyl dC, Inverted dT, Iso-dG, Iso-dC, Inverted Dideoxy-T, 5-Methyl dC, 5-Nitroindole, 5-hydroxybutynl-2′-deoxyuridine or 8-aza-7-deazaguanosine for example.

Complementary regions between modified or unmodified oligonucleotides may furthermore comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides with a sequence identity of at least 80% or 85%, preferably 95% and most preferably 100%. The term ‘complementary regions’ in this regard refers to parts or the whole of the modified or unmodified oligonucleotides.

The person skilled in the art is aware of means and methods to properly design said modified oligonucleotides that satisfy the aforementioned requirements.

The modified oligonucleotides and/or complexes in a non-limiting example may comprise two, three or four separate DNA oligonucleotides, which together comprise the following features: one or more 3′-lipid modification(s) (which in general can also be replaced by a cholesterol modification), a moiety for interlinking multiple complexes, a barcode sequence, a capture sequence preferably a polyadenylated 3′ end, for example as generally comprised in mRNA, a sequence that can serve as a primer binding site for subsequent PCR amplification, and hybridization sequences that allow oligos to anneal to each other. The skilled person is aware that the order of the sequence parts can be modified without abolishing the function of the oligonucleotide. Furthermore, more than four oligonucleotides can be assembled to result in even larger complexes.

In cases in which the modified oligonucleotides contain less than two oligonucleotides, annealing may not be necessary. In such an instance, the oligonucleotide may be linked to a lipid or cholesterol on its 3′ end and/or 5′end, and it may further encode a barcode sequence, a primer binding site, as well as a capture sequence, preferably a polyA sequence. Furthermore, the modified oligonucleotide may be an oligonucleotide with a length of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides whereas said nucleotides may also comprise oligonucleotide modifications such as phosphorothioated bases, phosphorothioated 2′ O-methyl bases, locked nucleic acids, 2-MethoxyEthoxy A, 2-MethoxyEthoxy MeC, 2-MethoxyEthoxy G, 2-MethoxyEthoxy T, 2′ Fluoro C, 2′ Fluoro A, 2′ Fluoro G, 2-Aminopurine, 5-Bromo dU, deoxyUridine, 2,6-Diaminopurine, Dideoxy-C, deoxylnosine, Hydroxymethyl dC, Inverted dT, Iso-dG, Iso-dC, Inverted Dideoxy-T, 5-Methyl dC, 5-Nitroindole, 5-hydroxybutynl-2′-deoxyuridine or 8-aza-7-deazaguanosine for example.

The oligonucleotide complex may be preassembled before being added to the group of cells/nuclei. The oligonucleotide complex can also be assembled subsequently, preferably at the membrane of the cells/nuclei. The (annealed) oligonucleotide(s) is/are added to the group of cells within the first reaction compartment and the oligonucleotide (complex) inserts into the cells' membrane via the modification as comprised in the modified oligonucleotide(s) thus enabling the group-specific barcoding of the group of cells. Alternatively, cells may be dissociated prior to addition of the barcode oligonucleotide(s) (complexes).

In another embodiment, individual cell groups being contained in separate first reaction compartments of a multiwell plate, may be barcoded with two or more distinct barcode oligonucleotide(s) (complexes), resulting in a unique set of barcodes that identifies cells originating from a group of cells. For instance, barcode combinations can be generated following a two-dimensional (2D) labelling scheme, similarly to what has been described before; see e.g. Gehring et al. (2019) Nat Biotechnol 38, 35-38 (2020);) see also Example 10.

In a further embodiment, it may be preferred that cell groups undergo successive rounds of split-pool group-specific barcoding (split-pool logic similar to Rosenberg et al. (2018) Science 360, 176-182), preferably using barcoding complexes containing one or several LMOs. In such an instance, cells belonging to the group of cells may be identified by a distinct combination of oligonucleotide complex associated barcodes in order to increase the throughput of cell groups that can be processed per experiment. In such an instance, cell groups may undergo consecutive cycles of a) isolation of multiple cell groups into a first reaction compartment, b) barcoding of cell groups, and c) pooling of barcoded cell groups. At the end of multiple of such a series of barcoding cycles, each cell group received a unique combination of barcodes, such that they can be pooled and completely dissociated.

As detailed above, within the present invention, the barcode sequence may also be attached to an antibody or another vehicle specific for a component of the cell/nuclei group, e.g. a surface protein or a component within the cell/nuclei.

The invention furthermore relates to interlinking of oligonucleotide complexes (LMOs/CMOs) on the cellular surface of individual cells of the group of cells, preferably under conditions that promote stable oligonucleotide attachment.

In certain embodiments of the invention, the oligonucleotide complex described herein comprises a moiety for interlinking two or more of the oligonucleotide complexes described herein. In further embodiments of the method of the invention the moiety for interlinking complexes can act directly, such as via photocrosslinking and/or chemical crosslinking groups, nucleophilic and electrophilic groups, multivalent molecules, protein-protein interactions, hybridizing bridge oligonucleotides, or indirectly via a multistep labeling procedure.

Interlinking of barcode oligonucleotide complexes may in general be achieved by any molecular interaction that relies on dipole-dipole interaction, electrostatic interaction, van der Waals force, hydrogen bonds, the hydrophobic effect, and/or covalent bonds.

In further embodiments of the invention, the method further comprises a step of adding an agent inducing the covalent or non-covalent interlinking of oligonucleotide complexes. The agent may be UV light, a hybridizing bridge oligonucleotide, a protein such as streptavidin, a chemical inducer, and/or click-chemistry.

In a preferred embodiment of the invention, interlinking and all subsequent steps until and including complete dissociation may be performed in the presence of an oligonucleotide complex (e.g. LMO/CMO) stabilizing agent.

Subsequent to the group-specific barcoding of the group of cells/nuclei, the method of the present invention comprises a step of completely dissociating said group of cells/nuclei in order to obtain barcoded single cells/nuclei. The skilled person is aware of means and methods that can be employed to completely dissociate the group of cells/nuclei within the methods of the present invention. In general, complete dissociation of the group of cells/nuclei may be carried out following one or a combination of the aforementioned methods comprising enzymatic, mechanical and/or chemical dissociation which are preferably carried out to an extent that ensures that all physical cell-cell interactions are broken.

In a preferred embodiment, barcoded cells/nuclei may be pooled prior or subsequent to complete dissociation in order to arrive at a single cell/nuclei suspension.

In a further step, the methods of the present invention comprise a cell/nucleus-specific barcoding of nucleic acid molecules contained in or attached to individual cells/nuclei from the group of cells/nuclei with a cell-specific barcode sequence.

Accordingly, the methods of the present invention comprise barcoding of nucleic acid molecules which may comprise DNA and/or RNA being contained in or attached to individual cells/nuclei. However, the methods of the present invention are not limited by the type of DNA or RNA being contained in said cells/nuclei. Thus, the DNA or RNA may be of any type known to the person skilled in the art. The DNA may preferably be genomic DNA and it may preferably represent parts or the entirety of the genome as comprised in the cells/nuclei used in the methods of the present invention. As such, the DNA comprised in the cells/nuclei is preferably in the form of autosomal/nuclear DNA and/or mitochondrial DNA. RNA may preferably represent parts or the entirety of the transcriptome as comprised in the cells used in the methods of the present invention, preferably the transcriptome in its entirety. As such, the RNA comprised in the cells is preferably in the form of messenger RNA (mRNA). As the skilled person will appreciate, mRNA generally comprises a polyadenylated (polyA) tail at its 3′ end and contain polyA stretches throughout the transcript. Accordingly, it may be preferred that the oligonucleotide comprising the group-specific barcode comprises a polyA sequence in order for the cell-specific barcode sequence of the invention to bind both, the cellular mRNA/DNA as well as the group-specific barcode.

Accordingly, barcoded single cells/nuclei being comprised of the cells/nuclei themselves as well as the group-specific oligonucleotide and/or oligonucleotide complex(es), for example being attached to the cells'/nuclei surfaces, are individually isolated into a second reaction compartment.

Said second reaction compartment may be a plastic tube, (microfluidic) droplet or a well in a multi-well plate. The multi-well plate may be a 96/384 well plate or a miniaturized multi-well plate. In another embodiment of the invention, the second reaction compartment may be generated by a microfluidic droplet generator. Within the present invention, both the first as well as the second reaction compartments may be standard plates. Exemplary plates include Seq-Well (Gierahn et al. (2017) Nature Methods 14, 395-8) or Microwell-Seq (Han et al. (2018) Cell 172, 1091-1107).

Isolation of cells/nuclei into the second reaction compartment may comprise the use of a microfluidic device, preferably a FACS/BACS instrument, more preferably a droplet generator or a microwell-based single-cell partitioning technology.

In a preferred embodiment, cells/nuclei may be lysed subsequent to isolation into the second reaction compartment. As such, the second reaction compartment may comprise lysed cells/nuclei. Lysis may be performed for example by heat denaturation and/or lysis buffers containing detergents such as Triton X-100, Triton-114, Tween-20, Tween-80, Brij-35, Brj-58, Octyl glucoside, Octyl thioglucoside, ammonium chloride, ethyl trimethyl ammonium bromide, NP40, CHAPS, CHAPSO, and/or sodium dodecyl sulfate (SDS).

In a non-limiting embodiment of the present invention, the cell/nucleus-specific barcode sequence may comprise at least three sequences being comprised in a single oligonucleotide, wherein a first sequence encodes for a primer binding site, a second sequence encodes for a barcode sequence and a third sequence encodes for a second primer binding site, preferably an oligoT-sequence to enable binding to polyA-sequences being present in mRNA or group-specific oligonucleotide(s) and/or complexes. A fourth sequence encoding a unique molecular identifier (UMI) may also be comprised.

Second barcoding of nucleic acid molecules within the second reaction compartment may be achieved by annealing and extension of the second primer binding site of the barcode sequence to its target region being contained in RNA and/or DNA oligonucleotides, whereas said oligonucleotides are released from the inner part of the cell/nuclei as well as from the membrane during lysis; subsequently, reverse transcription and second strand synthesis complete the second nucleic acid barcoding step (cDNA synthesis). The resulting extended cell/nuclei-specific barcode sequence encodes a cell/nuclei-specific barcode sequence. Accordingly, cDNA originating from the group-specific barcode sequence that encoded a group-specific first barcode, will encode two distinct barcodes (a group-specific as well as a cell/nuclei-specific barcode), whereas cDNA originating from cellular RNA or DNA encodes only one barcode (cell/nucleus-specific barcode). It should be noted however, that depending on the design of the barcode sequence, further (preferably unknown) barcode sequences may be incorporated into the elongated barcode sequences.

The present invention also relates to kits, in particular research kits. The kits of the present invention comprise the modified oligonucleotide(s) of the present invention preferably together with instructions regarding the use of the methods of the invention. The kits of the invention may further comprise an interlinking reagent and components for optimized ion concentrations that increase efficiency and stability of the membrane-barcoding.

In a particular embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the uses provided herein and in accordance with the present invention. Particularly, said instruction manual(s) may provide guidance to use or apply the herein provided methods or uses.

The kit (to be prepared in context) of this invention may further comprise substances/chemicals and/or equipment suitable/required for carrying out the methods and uses of this invention. For example, such substances/chemicals and/or equipment are reagents, solvents, diluents and/or buffers for stabilizing and/or storing and/or enabling enzymatic reactions or terminating enzymatic reactions, (a) compound(s) required for the uses provided herein, like stabilizing and/or storing the chemical agent(s) comprised in the kits of the present invention. In a preferred embodiment of the present invention, kits may, especially in view of successive rounds of repeated cell group barcoding by split-pool barcoding (Rosenberg et al. (2018) Science 360, 176-182) comprise ready to use multi-well, 96-well or 384-well microwell plates containing necessary barcoding reagents.

In a particular embodiment, the kit of the invention further comprises an agent inducing the covalent or non-covalent interlinking of complexes.

Further embodiments are exemplified in the scientific part. The appended figures provide for illustrations of the present invention. The experimental data in the examples and as illustrated in the appended figures are not considered to be limiting. The technical information comprised therein forms part of this invention.

The invention thus also covers all further features shown in the figures individually, although they may not have been described in the previous or following description. Also, single alternatives of the embodiments described in the figures and the description and single alternatives of features thereof can be disclaimed from the subject matter of the other aspect of the invention.

### BRIEF DESCRIPTION OF THE FIGURES

FIG. 1 (Example 1, 6). Improved membrane barcoding efficiency and stability with the new LMO barcoding strategy upon prolonged incubation at elevated temperature. A new lipid-modified oligonucleotide (LMO) barcoding (from hereon referred to as “new strategy”, see Example 1) is shown to perform better than the previous design of the prior art. Cells were barcoded with LMO-barcode complexes carrying a FAM-fluorescent barcode, using the original (violet) or the new (green) strategy. Upon 30 minutes at 4° C. (bright colors) the fluorescent intensity was ˜6-fold higher with the new compared to the original strategy. At 37° C. (dark colors), the loss of barcodes was also substantially reduced (loss of ˜54% versus ˜93%). Thus, even at elevated temperature the new LMO strategy achieves barcoding levels substantially higher than those previously achieved at 4° C. (dark green vs. light violet). Altering the ion concentration at 37° C. further significantly improved barcoding stability (orange). Importantly, while with the prior design, a considerable subset of cells had lost their LMO-barcode complexes, all cells remained barcoded with the new LMO strategy. Gray: non-barcoded cells (negative control).

FIG. 2 (Example 2, 7). Specificity of new LMO membrane barcoding strategy under dissociation conditions. Mouse embryonic stem cells were LMO-barcoded carrying a FAM-fluorescent barcode, using the improved barcoding strategy. LMO-barcoded cells were subsequently mixed with non-barcoded mCherry-expressing cells, and incubated at 37° C. in TrypLExpress for 30 minutes, mimicking strong dissociation conditions. mCherry-expressing cells did not acquire FAM-fluorescent barcodes as quantified by FACS analysis. Less than 0.5% of all cells were both, mCherry positive and LMO-FAM positive, indicating neglectable cross-labeling. Color indicates cell density.

FIG. 3 (Example 3, 8). High stability of the new LMO membrane-barcode strategy under dissociation conditions. LMOs carrying FAM-fluorescent barcodes were used to barcode single cells (blue, orange) using the new strategy. While one fraction was incubated at 4° C. (blue), the other was subjected to typical dissociation conditions (20 minutes at 37° C. in TrypLExpress and subjected to pipetting; orange). Only minimal FAM-barcode loss (˜16%) could be detected when both were compared. Gray: non-barcoded cells (negative control); green: cell group barcoding.

FIG. 4 (Example 4, 9). Sufficient membrane-barcoding of cells within cell groups. (A) Representative cell groups isolated after partial tissue dissociation of embryoid bodies (a multicellular in vitro model). The invention is compatible with a wide range of group sizes (2 to ˜200 cells). (B) The new LMO-barcoding strategy were used with FAM-fluorescent barcodes to barcode either single cells (orange) or cell groups (green). Upon cell group barcoding, cells were on average ˜30% less barcoded, compared to barcoding of single cells that had been subjected to the same dissociation conditions (TrypLExpress, 37° C., pipetting). This is not expected to prevent barcode identification as the reduced barcoding is still higher than what was achieved with the state-of-the-art LMO membrane-barcoding; (see also FIG. 1). Gray: non-barcoded cells (negative control).

FIG. 5 (Example 5, 10). LMO-barcodes can be used for the single-cell sequencing of cell groups from embryonic bodies (a multicellular in vitro model). Cells from cell groups were membrane-barcoded using the new strategy, single-cell RNA sequenced and subjected to dimensionality reduction based on their membrane barcodes (A) or transcriptomes (B, C). Groups of different size were isolated from joint, partial dissociation of embryoid bodies expressing either GFP or mCherry. Each cell group was dissociated into single cells and barcoded with a unique LMO barcode combination and then jointly subjected to scRNA-seq (10×Genomics 3′end scRNA-seq v3), which profiles the transcriptome and also reads out the membrane barcodes (i.e. the group-specific barcode) for each cell. (A) Cells are clustered based on LMO barcodes, revealing which cells originate from the same group. Each group contains either GFP- or mCherry-positive cells (green and red, respectively), indicating the absence of experimental impurities or computational misassignments. Transcripts revealing the type of expressed fluorophore were not detected in all cells (negative cells gray). Shown are cells from groups for which ≥5 cells were recovered. An example group is boxed and shown in transcriptional space in (C). (B) Same cells as in (A) but clustered based on their transcriptomes (numbers 0-7 label transcriptional clusters). The clustering indicates that the cell groups generated by partial dissociation contain cell with diverse transcriptional profiles. (C) Same cells as in (A) clustered based on their transcriptomes as in (B) with cells originating of one cell group shown in red as an example (relates to boxed cell group in A). The position of the red cells in different transcriptional clusters indicates that the cell group was composed of cells with distinct gene activities.

FIG. 6 (Example 10). Exemplary experimental outline for single-cell sequencing of interacting cells.

FIG. 7. Gating strategies can be designed to isolate cell groups by flow cytometry. (A) Schematic showing the relationship between particle size and its measured width and height. The time it takes for the particle to pass the laser indicates its height, a parameter that can be used to enrich cell groups. (B) Fluorescent activated cell sorting (FACS) gating strategy for isolating larger cell groups from a partially dissociated mouse embryo (embryonic day 11). Gates were chosen to select for live cells, from which the largest events were sorted using the forward scatter (FSC) and then their Hoechst signal. Hoechst stains DNA and can be used to identify events that comprise multiple nuclei. (C) Representative microscopy images of cell groups collected using the FACS gating strategy shown in (B). Scale bars, 100 μm.

FIG. 8 (Example 5, 12). LMO-barcodes can be used for the single-cell sequencing of cell groups from embryonic tissues.

Cells from cell groups were membrane-barcoded using the new strategy, single-cell RNA sequenced and subjected to dimensionality reduction based on their LMO barcodes (A) or transcriptomes (B, C). Groups of different size were isolated from partial dissociation of mouse embryos. Cell groups were membrane-barcoded with group-specific barcode combinations, subsequently jointly dissociated into single cells, and subjected to scRNA-seq (10×Genomics 3′end scRNA-seq v3), which profiles the transcriptome and also reads out the LMO barcodes (i.e. the group-specific barcode) for each cell. (A) Cells are clustered based on LMO barcodes, revealing which cells originate from the same group. The numerous clusters of cells in the barcode space reflects the number of uniquely barcoded cell groups. Shown are cells which were assigned to at least one out of four barcodes. An example group is boxed and shown in transcriptional space in (C). (B) Same cells as in (A) but clustered based on their transcriptomes (numbers 0-22 label transcriptional clusters). The clustering indicates that the cell groups generated by partial dissociation contain cell with diverse transcriptional profiles. (C) Same cells as in (A) clustered based on their transcriptomes as in (B) with cells originating of one cell group shown in red as an example (relates to boxed cell group in A). The position of the red cells in different clusters indicates that the cell group was composed of transcriptionally distinct cell types. (D) Comparison of the identified transcriptional clusters (colors) and their proportion from single-cell sequencing of normal single-cell dissociated mouse embryos and from partial dissociations that generate cell groups from mouse embryos for the purpose of cell group barcoding. The partial dissociation of mouse embryos recovers a broad spectrum of cell clusters (an approximation for diversity of cell type and cell lineages) and in largely similar proportions, indicating the absence of strong biases induced during partial dissociation.

Unless otherwise defined, all technical and scientific terms used herein have the same meaning as commonly understood by one of ordinary skill in the art to which the present invention pertains. Although methods and materials similar or equivalent to those described herein can be used in the practice or testing of the present invention, suitable methods and materials are described below. The materials, methods, and examples are illustrative only and not intended to be limiting.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d et., Cold Spring Harbor Laboratory Press, Cold Spring Harbor, N.Y. (1989) and Ausubel et al., Current Protocols in Molecular Biology. John Wiley & Sons (2003).

While the invention has been illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually, although they may not have been described in the afore or following description.

Furthermore, in the claims the word “comprising” does not exclude other elements or steps, and the indefinite article “a” or “an” does not exclude a plurality. A single unit may fulfil the functions of several features recited in the claims. The term “about” and the like in combination with an attribute or a value particularly also define exactly the attribute or exactly the value, respectively. Any reference signs in the claims should not be construed as limiting the scope.

### EXAMPLES

### Example 1

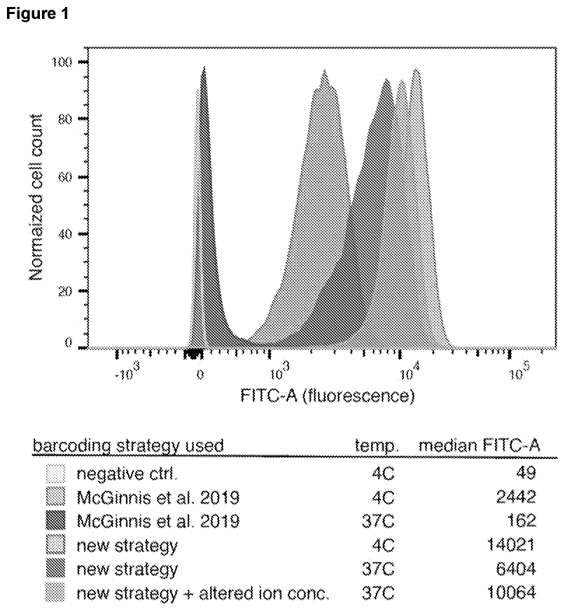

Specifically, in a first experiment, a new lipid modified oligonucleotide (LMO) barcoding strategy (from hereon referred to as “new strategy”) was shown to perform better than the prior art (McGinnis et al. (2019) Nat. Methods 16, 619-26) in terms of barcoding efficiency and stability/cell membrane retention (FIG. 1). In particular, the new strategy was based on DNA oligonucleotide complexes which incorporate the following features: 3′-lipid modification (which in general can also be replaced by a cholesterol modification), a moiety for interlinking multiple LMO complexes on the membrane, a barcode sequence, a polyA capture sequence, and a sequence that can serve as a primer binding site for PCR amplification. These LMO complexes integrated into the cell membrane, where their interlinking induces an increased stability/cell membrane retention. In contrast, the design of the state of the art contained two 3′ lipid (or cholesterol) modified oligonucleotides (anchor and co-anchor) that were partially complementary to each other as well as a barcode oligonucleotide containing a capture sequence and a primer binding site. Upon annealing and complex formation however, the LMO complexes of this previously published design could not be interlinked. Single cells were labelled with FAM-fluorescent barcode-LMO complexes with either the original or the new strategy and analysed after a certain incubation period. After 30 minutes on ice, cells barcoded with the new strategy showed surprisingly, a fluorescence intensity that was ˜6-fold higher with the new strategy compared to the previously published one due to improved barcode-LMO complex stability/membrane retention which is a direct consequence of the improved design. Furthermore, after 30 minutes at 37° C., cells showed a 93% loss of barcodes for the previously published barcoding compared to only 54% for the new barcoding strategy resulting in a ˜1.7-fold increased stability. Altering the concentration of free ions in the suspension surprisingly further improved stability and retention of barcodes, reducing their median loss to only 28% for the new strategy under the same barcoding conditions as were used before (FIG. 1). Thus, it was demonstrated that the new LMO strategy combined with an altered ion concentration of the suspension led to a significantly higher barcoding efficiency compared to the state-of-the-art LMO design and allowed the handling of oligonucleotide barcoded cells under conditions, where the prior-art LMO barcoding failed to preserve the cell's barcodes sufficiently.

### Example 2

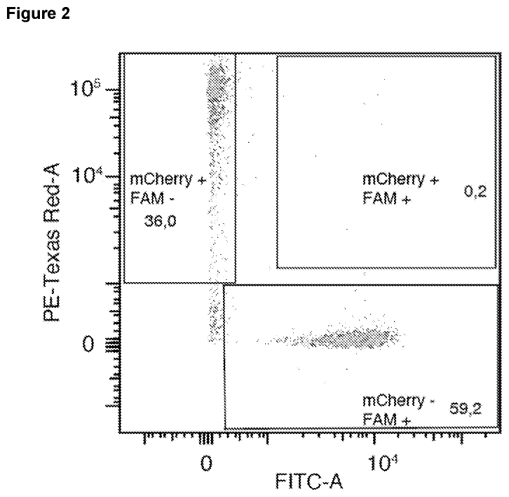

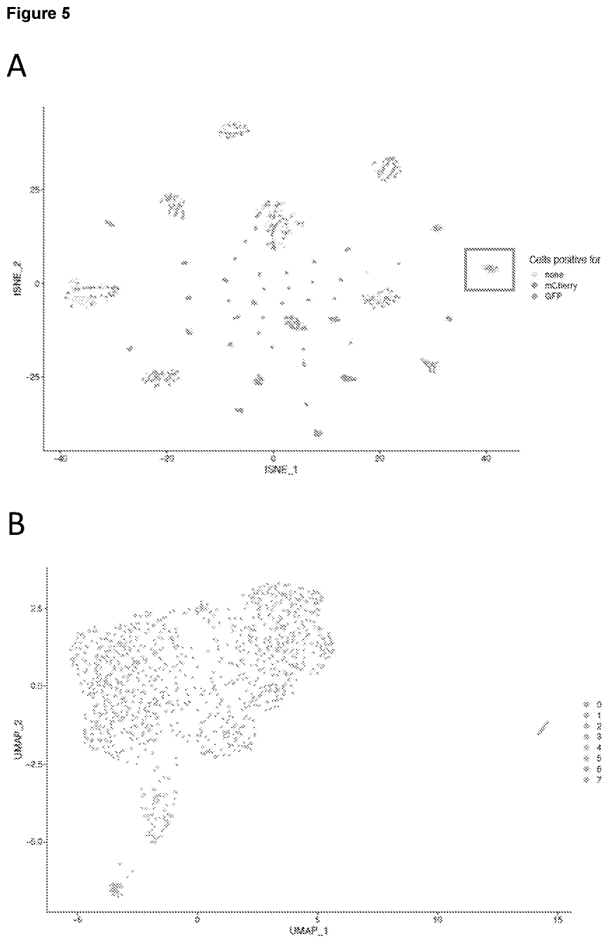

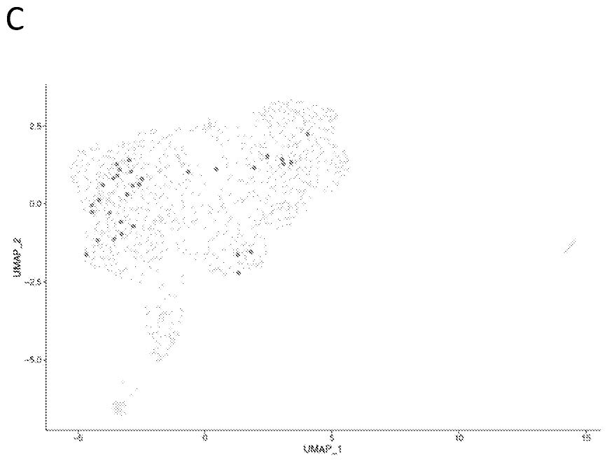

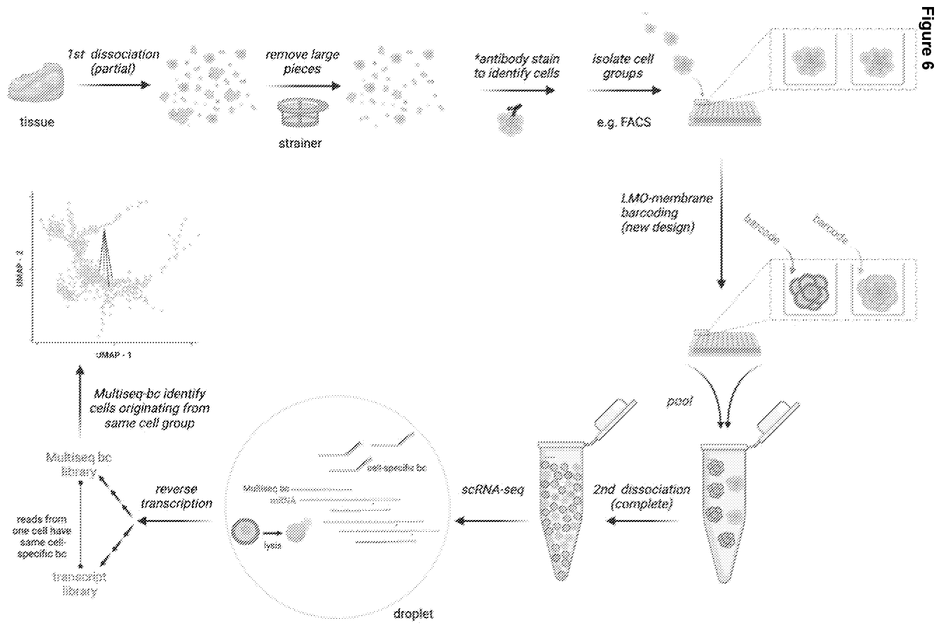

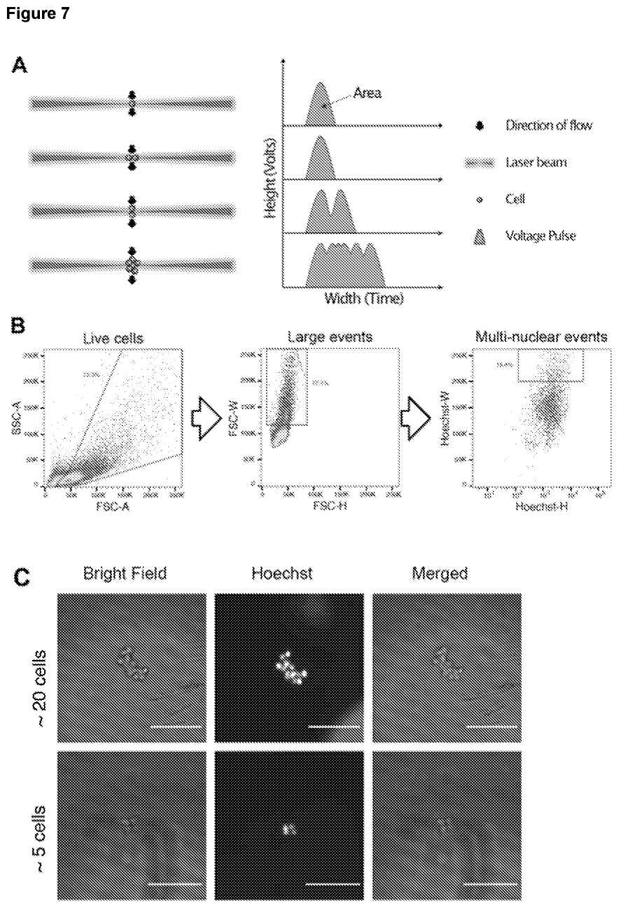

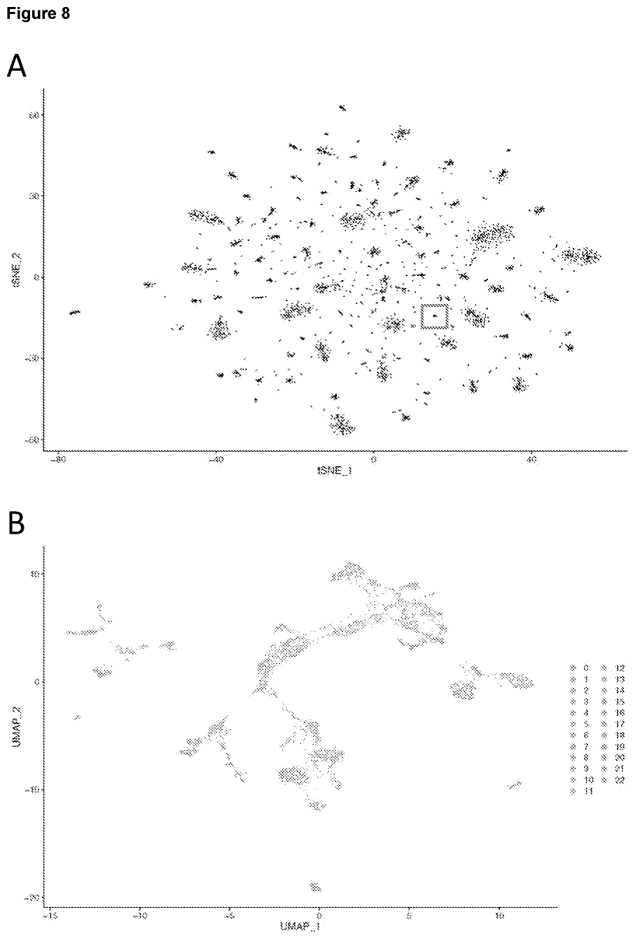

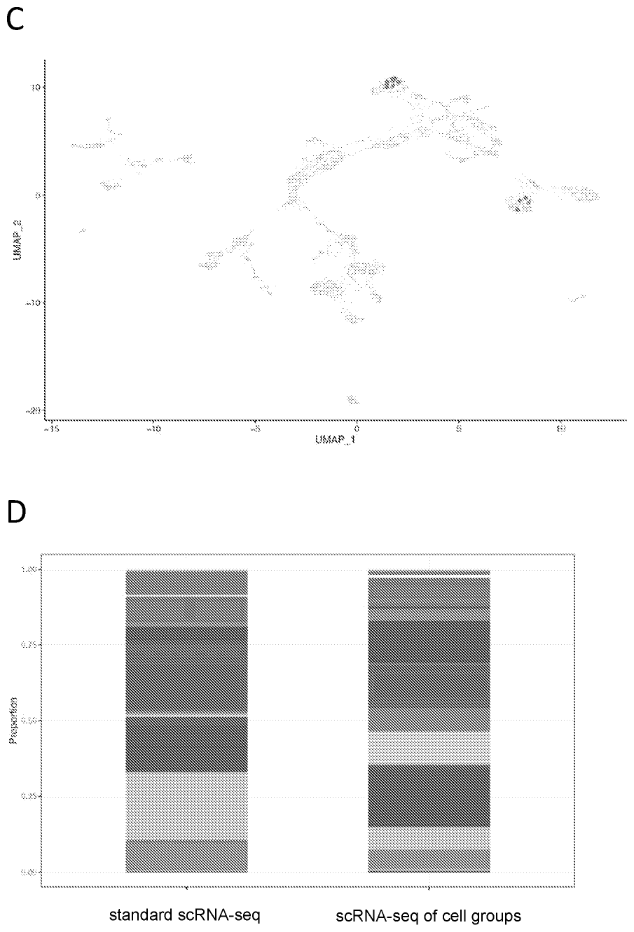

In a second experiment, it was shown that the new LMO strategy only leads to minimal barcode-LMO complex cross-talk between cells, even at elevated temperatures (FIG. 2). Single cells were barcoded with FAM-fluorescent barcode-LMO complexes of the new design and mixed with mCherry-expressing non-barcoded cells. The mixture was incubated under conditions reminiscent of complete dissociation (e.g. 30 minutes at 37° C. in TrypLExpress) and scanned using fluorescence activated cell sorting (FACS). It was demonstrated that the fraction of double-positive cells was close to zero, indicating high barcoding stability of the new strategy, which is crucial for reliable cell assignments. In most instances of the current invention, barcoded cells are completely dissociated by enzymatic and/or physical dissociation which requires more aggressive temperatures and/or buffers that were expected to compromise barcode-LMO complex retention in the membrane. It was unexpected that those barcode-LMO complexes that detach from the membrane do not reintegrate into another cell, which would have compromised the reliability of the LMO barcoding. It was therefore not obvious to combine LMO-barcoding of cells in cell groups with subsequent complete dissociation. Surprisingly, the new LMO strategy recovers highly barcoded cells (even from cell groups, see also FIGS. 5 and 8). While it was shown in the prior art that there is minimal LMO cross-talk between cells at room temperature (McGinnis et al. (2019) Nat. Methods 16, 619-26), the present results importantly furthermore indicate that the new LMO strategy provide high barcoding specificity, even under cell dissociation conditions.

### Example 3

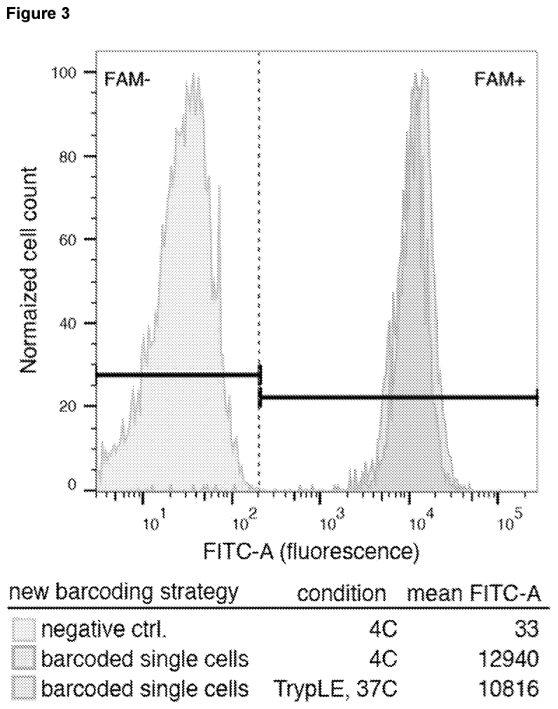

In a third experiment, it was similarly shown that the new strategy allows stable membrane-barcoding under dissociation conditions (FIG. 3). Single cells were barcoded using the new strategy with LMOs carrying FAM-fluorescent barcodes and then split into two fractions. While one fraction was incubated at 4° C., the other was subjected to typical dissociation conditions (20 minutes at 37° C. in TrypLExpress and subjected to several rounds of pipetting). Only minimal barcode loss (˜16%) could be detected during subsequent comparative analysis of both samples when cells were exposed to stringent dissociating conditions. Thus, it was demonstrated that potential disruptions of interlinked LMO complexes during the dissociation procedure is not causing increased barcode loss.

### Example 4

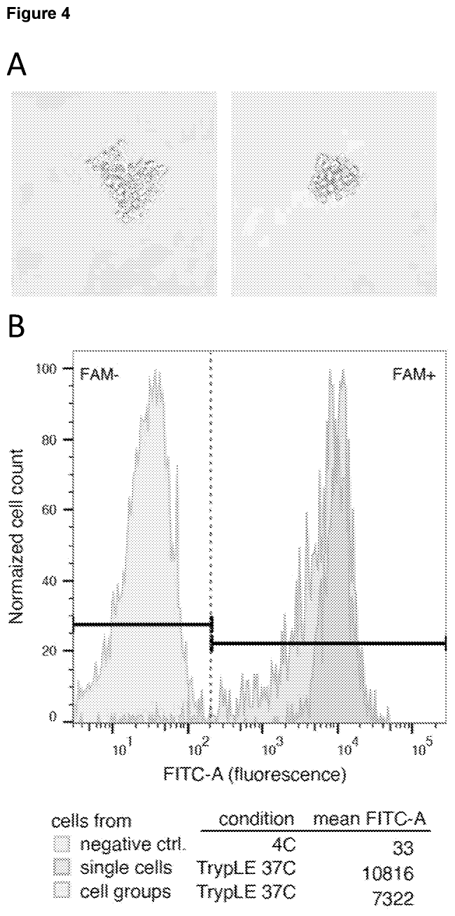

In a fourth experiment, it was furthermore shown that the new strategy can barcode groups of interacting cells sufficiently. In order to test, if innermost cells within a cell group could also be efficiently barcoded with the new barcoding strategy, LMO-complexes with FAM-fluorescent barcodes were added to cell groups of ˜50-150 physically attached cells. Subsequently, cells were completely dissociated into single cells and their fluorescent signal was analyzed. It was found that the vast majority of cells was barcoded, while LMO barcoding efficiency was on average only reduced by 30% and only a small subset of cells showed a ˜10-fold reduction (FIG. 4). This was unexpected as the extracellular matrix and adhesion molecules present between interacting cells could have prevented effective barcoding of cells. The successful labelling of interacting cells speaks for the fact that barcode-LMO complexes are small membrane labels, and in comparison to larger complexes such as antibodies, have a higher tissue penetrance. Since the cells closest to the center of larger cell groups were still barcoded to a degree comparable to the prior art (McGinnis et al. (2019) Nat. Methods 16, 619-26; see also FIG. 1), this enables the unambiguous identification of LMO/CMO-barcodes from sequencing data thus for the first time allowing the unbiased high-throughput transcriptomic profiling of interacting cells with single-cell resolution. It should be pointed out that this was made possible by generating cell groups of intact and viable cells, a result of partial dissociation. In contrast, approaches using mechanical tissue cutting are prone to damage the cells of smaller cell groups or the outermost cells of larger cell groups. Damaged cells at the surface of cell groups impede with effective LMO-barcoding as their membranes sequester LMO-barcodes and prevent efficient diffusion of LMO-barcodes in between cells. Thus, the present invention did overcome this limitation and achieves efficient LMO-barcoding of interacting cells, by combining partial dissociation and membrane-barcoding of resultant cell groups.

### Example 5

In a fifth set of experiments, it was demonstrated that RNA of cells originating from cell groups of an in vitro model (embryoid bodies) and of complex tissues (mouse embryo) can be sequenced and confidently identified using the new membrane-barcoding strategy. Embryoid bodies expressing either red or green reporter constructs were mixed, partially dissociated, and cell groups consisting of up to 200 cells were isolated into individual wells of a 96-well plate. Cell groups were completely dissociated and subsequently labeled with new barcode-LMO complexes (each well receiving a combination of two known barcodes). Subsequently, barcoded cells were pooled and single-cell RNA-seq libraries were constructed using the 10× Genomics technology suite (3′ scRNA-seq kit; Pleasanton, CA) in which an exemplary non-membrane bound, cell-specific barcode sequence of the present invention is contained to barcode all oligonucleotides being contained in or associated with a given single cell. After sequencing and during bioinformatic analysis, the combination of cell group specific LMO-barcodes as well as the cell-specific barcode which is introduced during single-cell library preparation both led to the surprising results provided herein. Specifically, the results of an exemplary LMO library preparation, sequencing experiment, data processing and filtering are depicted in FIG. 5 where interacting cells as well as their underlying transcriptomes could be concurrently identified in an unbiased manner for the very first time with single-cell resolution, a task that so far has remained unattempted in the prior art due to the lack of enabling cell group barcoding/preparation protocols. Since cells belonging to one cell group can only express either red or green reporter constructs, it was furthermore possible to verify the experimental and computational reliability of cell group identification and assignment (clustered cells of one group are either red or green in the depicted two-dimensional tSNE dimensionality reduction plot, see FIG. 5A), which was shown to be surprisingly reliable (no incorrect cell group assignment). Furthermore, it was demonstrated that the membrane-barcoding strategy also enables the analysis of cell groups of complex tissues, specifically mouse embryos. Here it was shown that increased stability of the new barcoding strategy allows for the barcoding of cell groups with subsequent dissociation into single cells. Here too, membrane barcodes indicate which cells were part of the same cell group (FIG. 8A), the transcriptomes of cells recovered in cell groups are diverse (FIG. 8B, D), and their analysis can identify which cell types are present within one cell group (example shown in FIG. 8C). Thus, it was demonstrated for the first time that the new barcoding strategy can be used for the concurrent unbiased transcriptomic profiling of physically interacting cells.

### Example 6

The following example illustrates the difference in cell LMO barcoding performance when the method known in the art (McGinnis et al. (2019) Nat. Methods 16, 619-26) is compared to the new method to which the present invention pertains (new oligonucleotide design, complex stabilization via interlinking and an altered ion concentration). Therefore, FAM fluorescently-modified barcode-LMO complexes of the previously published and the new strategy were used to barcode single cells following different barcoding conditions and barcoding efficiency was assessed by fluorescence-activated cell sorting (FACS).

### Sample Preparation

Cells were barcoded with LMO-barcode complexes comprising FAM fluorescently modified barcode oligonucleotide of the previously published and the new barcoding design to assess barcoding efficiency and stability under various conditions (Table 1; FIG. 1).

TABLE 1
Conditions to be tested.

| | Buffer during | | |
|---|---|---|---|
| LMO design | incubation | Temperature | |
| Unlabeled (negative control) | DPBS + 1% BSA | 4° | C. |
| New strategy | DPBS + 1% BSA | 4° | C. |
| New strategy | DPBS + 1% BSA | 37° | C. |
| New strategy | DPBS + 1% BSA, | 37° | C. |
| | altered ion | | |
| | concentration | | |
| Published (McGinnis et al. 2019) | DPBS + 1% BSA | 4° | C. |
| Published (McGinnis et al. 2019) | DPBS + 1% BSA | 37° | C. |

V6.5 mouse embryonic stem cells were grown in a 6-well plate and washed with 1×DPBS. Cells were dissociated by adding 500 μl of TrypLExpress and incubated at 37° C. for 7 min. Next, cells were pipetted to obtain a single cell suspension and TrypLE was inactivated by adding 2 ml of DPBS. The suspension was subsequently transferred into a 15 ml Falcon containing 8 ml DPBS and cells were centrifuged for 5 min at 200 g. Supernatant was removed and the cell pellet was resuspended in 1 ml DPBS. Cells were counted, resuspended to a concentration of 1120 cells/μl.

Barcoding reactions for the new and the previously published LMO design were prepared as follows:

Components of LMO-complexes containing a FAM-Barcode were mixed each with a final concentration of 2 μM and incubated for 5 min at room temperature. For each condition to be tested (Table 1), 180 μl (˜2E5 cells) of the cell suspension were transferred into a LoBind Eppendorf tube containing the prepared oligonucleotide mix to a final barcoding volume of 200 μl. Samples were mixed by pipetting and incubated on ice for 5 min, an interlinking agent was supplied during an incubation on ice for 30 min. 1 ml of 1% BSA in DPBS was added and samples were mixed by pipetting. Cells were centrifuged at 200 g for 5 min at 4° C. and supernatant was removed. This washing step was repeated twice. The previously published LMO-barcoding was performed as described in McGinnis et al.

Samples were subsequently subjected to different temperatures and buffers to measure stability: according to Table 1, samples were resuspended in either 200 μl of DPBS+1% BSA or of DPBS+1% BSA+a chelator, and incubated at either 4° C. or 37° C. for 30 min. Cells were washed by adding 1 ml of DPBS+1% BSA and samples were centrifuged at 300 g for 5 min at 4° C. Supernatant was discarded and cells were resuspended in 200 μl of DPBS+1% BSA before subjecting cells to FACS analysis.

### Results

After 30 min of incubation on ice, the fluorescent intensity was ˜6-fold higher with the new design (light green) compared to the one of the state-of-the-art (light violet) due to improved LMO-barcoding efficiency/membrane retention (FIG. 1). Likewise, after 30 min at 37° C. cells showed a 93% median barcode-LMO loss with the previously published design (dark violet), resulting in low overall barcoding and a fraction of cells that had lost their LMO barcoding completely. In contrast, with the new strategy, the loss was only about 54% (˜1.7-fold increased stability; dark green), keeping all cells highly barcoded. Furthermore, the inventors discovered that altering the ion concentration in the cell suspension improves LMO-barcode stability/retention, further reducing the median LMO-barcode loss to only 28% (orange).

Thus, in summary, this example proves that the new barcoding strategy and modifications to the protocol enables significantly more efficient LMO-based barcoding of cells under conditions (i.e., elevated temperatures) where the previously published LMO strategy of the state of the art provided insufficient results.

### Example 7

The following example illustrates the negligible barcode-LMO cross-talk between barcoded and non-barcoded cells under dissociation conditions, which is crucial for reliable sequencing based cell assignment.

### Sample Preparation

Single V6.5 mouse embryonic cells were barcoded with FAM-fluorescent barcode-LMO complexes of the new LMO-strategy as described above (as in Example 6). Subsequently, cells were mixed with mCherry-expressing unlabeled cells and incubated under conditions reminiscent of dissociation (30 minutes at 37° C. in TrypLExpress). Subsequently cells were washed and prepared for FACS analysis as described in Example 6.

### Results

FAM-fluorescent and mCherry-fluorescent cells were separated by FACS and the fraction of “false” double positive cells was assessed to be close to zero (FIG. 2), indicating that the risk of LMO-barcodes detaching from one cell and attaching to another cell is negligible, which is crucial for reliable cell assignments. The two fluorescent groups (FAM and mCherry) were chosen to minimally overlap in excitation and emission, allowing for detection of signals by FACS in two entirely different channels. Importantly, while the prior art (McGinnis et al. (2019) Nat. Methods 16, 619-26) was only able to show that there is minimal LMO cross-talk between cells for the previously published LMO design at room temperature, the experimental results of Example 7 clearly indicate that the new LMO barcoding strategy provides high barcoding specificity, even under cell dissociation conditions.

### Example 8

The following example illustrates that enzyme treatment of LMO barcoded cells does not lead to a significant barcode-LMO loss, indicating the high barcoding stability under dissociation conditions using the new barcode-LMO architecture/protocol.

### Sample Preparation

Single V6.5 mouse embryonic cells were labeled with FAM-fluorescent LMO-barcodes of the new strategy as described above (as in Example 6) and split into two fractions. While one fraction remained at 4° C., the other one was subjected to typical dissociation conditions (20 min at 37° C. in TrypLExpress and subjected to several rounds of pipetting). Subsequently cells were washed and prepared for FACS analysis as described in Example 6.

### Results

Comparative FACS analysis of both samples detected only minimal LMO-barcode loss (˜16%; blue versus orange populations) when cells were exposed to stringent dissociation conditions (FIG. 3), highlighting that potential disruptions of interlinked LMO complexes during the dissociation procedure is not causing increased LMO loss.

### Example 9

The following example illustrates that cells within cell groups can be membrane-barcoded sufficiently, which is especially important for larger cell groups of e.g. 100 cells, where the innermost cells might be less exposed to LMO barcoding.

### Sample Preparation

Cell groups were generated by partially dissociation embryoid bodies grown for 5 days from mouse embryonic stem cells. Partial dissociation was achieved by incubating embryoid bodies for 4 minutes at 37° C. followed by four gentle pipetting steps with a 1 ml pipette. Subsequently single cells were removed and cell groups enriched using a 40 μm strainer. Cell groups of ˜50-150 cells were isolated into individual wells of a 96-well plate by manual pipetting. FAM-fluorescent barcode-LMO complexes of the new design were used to barcode cell groups while their cells were still attached to one another (as in Example 6 but downscaled to a lower barcoding volume per well and cell group). Subsequently, cell groups were dissociated into single cells (TrypLExpress, 20 minutes at 37° C., repeated pipetting). Subsequently cells were washed and prepared for FACS analysis as described in Example 6.

### Results

It was found that (i) most of the cells were barcoded, that (ii) the LMO barcoding efficiency was on average reduced by 30% compared to single cells that were barcoded using the same labelling protocol and that (iii) only a small subset of cells showed a ˜10-fold reduction in barcoding efficiency (FIG. 4). It can be assumed that the cells closest to the center of lager cell groups experienced reduced LMO barcoding. However, these cells were still barcoded to a degree comparable to the state of the art (McGinnis et al. (2019) Nat. Methods 16, 619-26), which should allow the unambiguous LMO barcode identification from sequencing data. Thus, it was shown that LMO barcoding can be used to sufficiently barcode cells within groups of cells which should allow the sequencing of interacting cells (for further information on single-cell RNA-sequencing (scRNA-seq) of cell groups see also Examples 5, 10, 12).

### Example 10

The following example illustrates that cells of cell groups from multicellular in vitro models can be membrane-barcoded with the new strategy after isolating individual groups and fully dissociating them into single cells. scRNA-seq of the membrane-barcoded cells profiles their transcriptomes and identifies which cells had been part of the same cell groups based on their membrane barcodes.

### Sample Preparation

Embryoid bodies expressing either mCherry or GFP transcripts were together partially dissociated (as described in Example 9) and cell groups consisting of ˜100-200 cells were pipetted into individual wells of a 96-well plate. Cell groups were completely dissociated (TrypLExpress, 20 minutes at 37° C., repeated pipetting) and subsequently barcoded with new LMO-barcoding strategy, each well receiving a different combination of barcode-LMO complexes of two distinct barcode sequences (as in Example 6 but downscaled barcoding volume per well and cell group). The usage of a combination of two barcodes per cell group was tested to confirm that the experimental and computational steps allow for the reliable identification of barcode combinations. A standard scRNA-seq library as well as a separate LMO barcode library were constructed using a slightly modified version (custom bead-based purification steps for LMO barcode library) of the 10× Genomics 3′ kit (Pleasanton, CA) following the manufacturer instructions and sequenced on the NovaSeq 6000 Platform.

### Results

Cells belonging to the same cell group could be identified based on identical LMO barcode combinations. Since cell groups had been generated from embryoid bodies expressing either mCherry (red) or GFP (green) reporter constructs, the inventors were able to verify the experimental and computational reliability of cell group identification and assignment (a cell group contained cells expressing either mCherry or GFP-constructs but not both) while at the same time extracting transcriptome information (FIG. 5). Thus, this example proves that LMO barcoding can efficiently be used for single-cell RNA sequencing of interacting cells within cell groups.

### Example 11

The following example illustrates one exemplary complete workflow for the single-cell sequencing of interacting cells.

An exemplary experimental workflow for the single-cell sequencing of interacting cells is depicted in FIG. 6. The tissue is partially dissociated with a reagent and procedure suitable for the tissue type and performed to the extent that most cells of interest are still present in small cell groups (both of which is known to the person skilled in the art).

The suspension can subsequently be passed through a strainer with a mesh size that removes tissue pieces that are too large (e.g. 100 μm mesh size). Cell groups can be specifically sorted by FACS based on fluorescent reporter model systems or cell surface markers, granularity, and/or DNA staining (e.g. Hoechst) to distinguish relevant cell groups from single cells. Each cell group is subsequently isolated for example into an individual well of a 96- or 384-well plate (first reaction compartment) and individual cell groups are membrane-barcoded using the new barcode-LMO strategy. In this regard, each cell group can be barcoded with either one or a combination of unique barcodes which can drastically increase the throughput via adoption of a split-pool barcoding scheme (Rosenberg et al. (2018) Science 360, 176-182). Subsequently, barcoded cell groups are pooled, completely dissociated into single cells and subjected to single cell library preparation.

Since the LMO barcode is compatible with 10× Genomics' 5′ and 3′ kits (Pleasanton, CA) and other prior art sequencing platforms as are listed in the above, single-cell sequencing libraries can be constructed using the transcriptome of a cell and the attached LMO barcode oligonucleotides. Briefly, in droplet based scRNA-seq, single cells are encapsulated into droplets (second reaction compartment) together with agarose beads carrying uniquely barcoded reverse transcription primers (cell-specific barcode sequence of the present invention). Within a droplet, the cell is lysed which releases the cellular mRNA repertoire as well as the LMO barcodes into the droplet. The droplet/cell-specific barcode sequence is also released from the agarose bead and binds with its oligo-dT-sequence to the polyA-stretches of mRNAs and LMO barcodes and initiates reverse transcription. Thus, reverse transcription of the LMO barcode results in amplicons containing information of both, the first LMO barcode as well as the second cell specific barcode which can then be used for cell group assignment during data analysis.

### Example 12

The following example illustrates that cells of cell groups from tissue of in vivo model systems can be membrane-barcoded with the new strategy, and that the membrane barcoding of cells can be performed when cells are still present in cell groups, before jointly dissociating barcoded cell groups in into single cells. scRNA-seq of the membrane-barcoded cells profiles their transcriptomes and identifies which cells had been part of the same cell groups based on their membrane barcodes.

### Sample Preparation

Mouse embryos of embryonic day 9.5 were partially dissociated and cell groups consisting of ˜5-200 cells were membrane-barcoded with group-specific combination of barcodes. Cell groups were completely dissociated (TrypLExpress, 20 minutes at 37° C., repeated pipetting). A standard scRNA-seq library as well as a separate LMO barcode library were constructed using a slightly modified version (custom bead-based purification steps for LMO barcode library) of the 10× Genomics 3′ kit (Pleasanton, CA) following the manufacturer instructions and sequenced on the NovaSeq 6000 Platform.

### Results

For most cells (>80%) one or more barcodes could be assigned with the chosen sequencing depth. Dimensionality reduction of these cells based solely on their sequenced membrane-barcodes clusters cells of similar barcode profiles together (barcode space, FIG. 8A). Analysis of the cell transcriptomes revealed the recovery of diverse cell types when sequencing cells from cell groups (FIG. 8B). Cells identified to originate from the same group can be analyzed for their gene expression and can, for instance, uncover cell groups composed of distinct cell types (example of one cell group shown in FIG. 8C). Further, the vast majority of cell types identified by standard scRNA-seq of mouse embryos, is also recovered in cell group sequencing, largely in similar proportions (FIG. 8D). Thus, this example proves that LMO barcoding can efficiently be used for single-cell RNA sequencing of interacting cells within cell groups from tissues and in vivo models.

Altered library preparation to read out LMO-bound barcodes in single-cell RNA sequencing (as published by McGinnis et al. 2019)

Cells carrying or containing cell-group specific barcodes are subjected to single-cell RNA sequencing. In most single-cell technologies cells are isolated into separate compartments where they are lysed, and the released mRNAs are reverse transcribed from primers containing cell-specific barcodes. Also, LMO-bound barcode sequences are released from the cell's membrane, bound by the cell-specific barcode primer which hybridizes to the poly A and initiate the reverse transcription.

Of the reverse transcription of mRNAs and cell-group-specific barcodes, a second priming site is integrated into the cDNA by a template-switching step. cDNAs are purified and amplified by PCR. To improve the amplification of the group-specific barcode, an additional primer can be optionally added, which will allow amplification even for templates that have not undergone successful template-switching. The amplified cDNA is purified in two steps, allowing for the separate and efficient recovery of transcript cDNA and membrane-barcode cDNA (i.e. here group-specific barcodes), as described in McGinnis et al. 2019. Specifically, 0.6 volumes of SPRI clean-up beads are added, and the supernatant is not disposed but instead used to recover the membrane-barcode cDNA amplicons (i.e. here group-specific barcodes cDNA amplicons), while the endogenous transcript cDNA amplicons are bound to the beads further processed according to the standard scRNA-seq protocol. The membrane-barcode cDNA amplicons (i.e. here group-specific barcode cDNA amplicons) are purified using a high amount of SPRI beads and isopropanol, as described for MULTI-Seq barcodes by McGinnes et al. 2019. Two sequencing libraries are prepared: one for sequencing the endogenous transcripts of cells (here the cell-specific barcode is connected with the information of transcripts) and one for the sequencing of group-specific barcodes (here the cell-specific barcode is connected with information on the group barcodes). The resultant data thus reveals for each cell a profile of its transcriptome plus the information which cell group it originated from.

### CITED NON PATENT LITERATURE

[NPL1] Rozenblatt-Rosen, O., Stubbington, M. J. T., Regev, A. & Teichmann, S. A. (2017). The Human Cell Atlas: from vision to reality. Nature, 550, 451-453.
[NPL2] Picelli, S., Faridani, O. R., Björklund, A. K., Winberg, G., Sagasser, S. & Sandberg, R. (2014). Full-length RNA-seq from single cells using Smart-seq2. Nat Protoc. 9, 171-181.
[NPL3] Macosko, E. Z., Basu, A., Satija, R., Nemesh, J., Shekhar K., Goldman, M., . . . & McCarroll, S. A. (2015). Highly parallel genome-wide expression profiling of individual cells using nanoliter droplets. Cell 161, 1202-1214.
[NPL4] Zheng, G. X. Y., Terry, J. M., Belgrader P., Ryvkin, P., Bent, Z. W., Wilson, R., . . . & Bielas, J. H. (2017). Massively parallel digital transcriptional profiling of single cells. Nat Commun. 8, 14049.
[NPL5] Rosenberg, A. B., Roco, C. M., Muscat, R. A., Kuchina, A., Sample, P., Yao, Z., . . . & Seelig, G. (2018). Science 360, 176-182.
[NPL6] Efremova, M., Vento-Tormo, M., Teichmann, S. A. & Vento-Tormo, R. (2020). CellPhoneDB: inferring cell-cell communication from combined expression of multi-subunit ligand-receptor complexes. Nat Protoc. 15, 1484-1506.
[NPL7] Stickels, R. R., Murray, E., Kumar, P., Li, J., Marshall, J. L., Di Bella, D. J. . . . & Chen, F. (2021). Highly sensitive spatial transcriptomics at near-cellular resolution with Slide-seqV2. Nat Biotechnol. 39, 313-319.
[NPL8] Lohoff, T., Ghazanfar, S., Missarova, A., Koulena, N., Pierson, N., Griffiths, J. A., . . . & Marioni, J. C. (2020). Highly multiplexed spatially resolved gene expression profiling of mouse organogenesis. bioRxiv. doi: https://doi.org/10.1101/2020.11.20.391896
[NPL9] Boisset, J.-C., Vivié, J., Grün, D., Muraro, M. J., Lyubimova, A. & van Oudenaarden, A. (2018). Mapping the physical network of cellular interactions. Nat Methods 15, 547-553.
[NPL10] Giladi, A., Cohen, M., Medaglia, C., Baran, Y., Li, B., Zada, M. . . . & Amit, I. (2020). Nat Biotechnol. 38, 629-637.
[NPL11] Manco, R., Averbukh, I., Porat, Z., Halpern, K. B., Amit, I. & Itzkovitz, S. (2021). Nat Commun. 12, 3074.
[NPL12] McGinnis, C. S., Patterson, D. M., Winkler, J., Conrad, D. N., Hein, Srivastava, V. . . . & Gartner Z. J. (2019). MULTI-seq: sample multiplexing for single-cell RNA sequencing using lipid-tagged indices. Nat Methods 16, 619-626.
[NPL13] Stoeckius, M., Zheng, S., Houck-Loomis, B., Hao, S., Yeung, B. Z., Mauck 3^{rd}, W. M. . . . & Satija, R. (2018). Cell hashing with barcoded antibodies enables multiplexing and doublet detection for single cell genomics. Genome Biol. 19, 224.
[NPL14] Busse, C. E., Czogiel, I., Braun, P., Arndt, P. F. & Wardemann, H. (2014). Single-cell based high-throughput sequencing of full-length immunoglobulin heavy and light chain genes. Eur J Immunol. 44, 597-603.
[NPL15] Gierahn, T. M., Wadsworth 2^{nd}, M. H., Hughes, T. K., Bryson, B. D., Butler, A., Satija, R. . . . & Shalek, A. K. (2017). Seq-Well: portable, low-cost RNA sequencing of single cells at high throughput. Nat Methods 14, 395-398.
[NPL16] Han, X., Wang, R., Zhou, Y., Fei, L., Sun, H., Lai, S. . . . & Guo, G. (2018). Mapping the mouse cell atlas by Microwell-Seq. Cell 172, 1091-1107.
[NPL17] Gillam, S. & Smith, M. (1979). Site-specific mutagenesis using synthetic oligodeoxyribonucleotide primers: I. Optimum conditions and minimum ologodeoxyribonucleotide length. Gene 8, 81-97.
[NPL18] Innis, M. A. (1990). PCR protocols: a guide to methods and applications. Academic Press, San Diego, USA.
[NPL19] Goulian, M., Goulian, S. H., Codd, E. E. & Blumenfield, A. Z. (1973). Properties of oligodeoxynucleotides that determine priming activity with Escherichia coli deoxyribonucleic acid polymerase I. Biochemistry 12, 2893-2901.
[NPL20] Sambrook, J., Fritsch, E. R., & Maniatis, T. (1989). Molecular Cloning: A Laboratory Manual (2^{nd} ed.). Cold Spring Harbor, NY: Cold Spring Harbor Laboratory Press.
[NPL21] Ausubel, F. M., Brent, R., Kingston, R. E., Moore, D. D., Seidman, J. G., Smith, J. A. & Struhl, K. (eds) (2003). Current Protocols in Molecular Biology. John Wiley & Sons.

All non-patent documents cited herein are hereby incorporated by reference in their entirety.

1. A method for obtaining sequencing-based information from cells/nuclei, the method comprising
(a) a first barcoding of cells and/or nuclei of a group of cells and/or nuclei, which comprises interacting cells with one or more group-specific barcode sequence(s) or a group-specific combination of two or more barcode sequence(s);
(b) a second barcoding of nucleic acid molecules contained in and/or attached to individual cells and/or nuclei from said group of cells/nuclei with a cell/nucleus-specific barcode sequence; and
(c) sequencing of the barcoded nucleic acid sequences.

2. The method of claim 1, wherein prior to step (a) the method comprises isolation of a group of cells/nuclei to be barcoded from a larger group of cells.

3. The method of claim 2, wherein the larger group of cells is partially dissociated to obtain the group of cells and/or nuclei used in step (a).

4. The method of claim 3, wherein partial dissociation is achieved by mechanical, chemical and/or enzymatic dissociation.

5. The method of claim 4, wherein the obtained group of cells/nuclei comprises 2 to 100, 2 to 200, 2 to 300, 2 to 400, 2 to 500, 2 to 600, 2 to 700, 2 to 800, 2 to 900, 2 to 1000, 2 to 5000, 2 to 10000 or 2 to 20000 cells/nuclei.

6. The method of claim 5, wherein the obtained group of cells/nuclei comprises 2 to 100 cells/nuclei.

7. The method of claim 1, wherein in step (a) one or more membrane-bound group-specific barcode sequence(s) is/are used.

8. The method of claim 7, wherein in step (a) one or more nucleic acid sequence(s) comprising a group-specific barcode sequence or a group-specific combination of two or more barcode-sequences are comprised of or complexed in/with (a) a modified primary oligonucleotide(s).

9. The method of claim 1, wherein in step (a) one or more barcoding antibody(ies) are used or a group-specific combination of two or more barcoding antibody(ies), wherein the barcoding antibody or the group-specific combination thereof comprises a group-specific barcode.

10. The method of claim 1, wherein in step (a) one or more nucleic acid sequence(s) comprising a group-specific barcode sequence or a group-specific combination of two or more barcode-sequences are used, wherein the nucleic acid sequence(s) are internalized into cells/nuclei.

11. The method of claim 1, wherein in step (a) one or more nucleic acid sequence(s) comprising a group-specific barcode sequence or a group-specific combination of two or more barcode-sequences is/are used, wherein the nucleic acid sequence(s) are modified to bind covalently or non-covalently to cellular proteins, transmembrane proteins or extracellular components.

12. (canceled)

13. The method of claim 8, wherein the group-specific barcode sequence is contained in an oligonucleotide complex comprising at least one oligonucleotide on the cellular surface of individual cells/nuclei of the group of cells/nuclei.

14. The method of claim 13, wherein the oligonucleotide complex further comprises a moiety for interlinking said complexes.

15. The method of claim 14, wherein the moiety for interlinking complexes can act directly or indirectly via a multistep labeling procedure.

16. The method of claim 14, further comprising a step of adding an agent inducing the covalent or non-covalent interlinking of complexes.

17. The method of claim 1, which is performed in the presence of an agent that increases barcoding efficiency, stability, and/or retention within and/or attached to the cells/nuclei.

18. The method of claim 1, wherein the group of cells/nuclei is generated by
(a) allowing individual cells to interact with each other; or
(b) mechanical dissociation, laser dissection, enzymatic dissociation, chemical dissociation, and/or cutting.

19. The method of claim 1, wherein the group of cells/nuclei are isolated by manual isolation and/or automated isolation.

20. The method of claim 19, wherein said isolation comprises the use of a fluorescent cellular reporter protein, fluorescent staining, fluorescent DNA staining and/or fluorescent antibody staining.

21. The method of claim 1, wherein the cells/nuclei prior to step (b) were subject to a non-natural influence.

22. The method of claim 1, wherein prior to step (b) cells/nuclei were contacted with a chemical compound, a biological cell, a pathogen and/or a virus.

23. The method of claim 1, wherein the cells/nuclei have been obtained from an organism, embryo, fetus, tissue, organ, body fluid, tumor, and/or an in vitro culture.

24. The method of claim 1, wherein the cells comprise primary cells, genetically engineered cells, blood cells, somatic cells, natural or altered immune cells, virus-infected cells, pluripotent stem cells (iPS), embryonic stem cells, epiblast stem cells, adult stem cells, embryoid bodies, gastruloids, organoids, in vitro embryo models, and/or cell lines, and wherein the nuclei comprise nuclei obtained from one or more of said cells.

25. The method of claim 1, wherein sequencing is performed using Drop-seq, DroNc-seq, Nx1-seq, Seq-Well, sci-RNA-seq, scifi-RNAseq, sci-RNA-seq3, SCRB-seq, CEL-Seq, CEL-Seq2, SPLIT-seq, MARS-seq, Smart-seq/C1, C1-CAGE, RamDa-seq, Microwell-seq, Smart-seq2, Smart-seq3, bead-seq, LIBRA-seq, G&T-seq, Quartz-Seq, Perturb-seq, MULTI-seq, ChIP-seq, ATAC-seq, SNES, scBS-seq, scRRBS, Smart-RRBS, Drop-ChIP, scDam&T-seq, ScNaUmi-seq, DR-seq, CITE-seq, TotalSeq, CytoSeq, STRT-seq, STRT/C1, SUPeR-seq, REAP-seq, MATQ-seq, scM&T-seq, scMT-seq, scCAT-seq, Paired-seq, SNARE-seq, T-ATAC-seq, Slide-seq, RAGE-Seq, FB5P-seq, TEA-seq, SUGAR-seq, CRISP-seq, single-cell RNA-seq, single-cell sequencing using Fluent, multiplexed scRNA-seq and/or CROP-seq.

26. An oligonucleotide complex comprising an oligonucleotide linked to a hydrophobic entity, a barcode sequence, and a moiety for interlinking complexes.

27. The oligonucleotide complex of claim 26, wherein the moiety for interlinking complexes can act directly or indirectly via a multistep labeling procedure.

28. (canceled)

29. A kit comprising the oligonucleotide and/or the oligonucleotide complex of claim 26.

30. The kit of claim 29 further comprising an agent inducing the covalent or non-covalent interlinking of complexes.
